(19) United States
(12) Patent Application Publication
(10) Pub. No.: US 2003/0219734 A1
(43) Pub. Date: Nov. 27, 2003

(54) Polypeptides related to natriuretic peptides and methods of their identification and use

(75) Inventor: Kenneth F. Buechler, Rancho Santa Fe, CA (US)

Correspondence Address: FOLEY & LARDNER, P.O. BOX 80278, SAN DIEGO, CA, 92138-0278, US

(73) Assignee: Biosite Incorporated

(21) Appl. No.: 10/419,059

(22) Filed: Apr. 17, 2003

Related U.S. Application Data
(63) Continuation-in-part of application No. 09/835,298, filed on Apr. 13, 2001.
(63) Continuation-in-part of application No. PCT/US02/26604, filed on Aug. 20, 2002.
(63) Continuation-in-part of application No. 10/139,086, filed on May 4, 2002.
(60) Provisional application No. 60/313,775, filed on Aug. 20, 2001.
(60) Provisional application No. 60/334,964, filed on Nov. 30, 2001.
(60) Provisional application No. 60/346,485, filed on Jan. 2, 2002.
(60) Provisional application No. 60/288,871, filed on May 4, 2001.
(60) Provisional application No. 60/315,642, filed on Aug. 28, 2001.

(51) Int. Cl. C12Q 1/70; G01N 33/53; G06F 19/00; G01N 33/48; G01N 33/50; G01N 33/543

(52) U.S. Cl. 435/5; 435/7.1; 436/518; 702/19

(57) ABSTRACT

The present invention relates to the identification and use of polypeptides that bind to antibodies directed to a desired polypeptide of interest. Using natriuretic peptides and their precursors, and in particular BNP, as an example, the present invention describes a number of natriuretic peptides fragments produced in biological samples, most preferably blood-derived samples, that bind to antibodies directed to BNP. Because production of such fragments is an ongoing process that may be a function of, inter alia, the elapsed time between onset of an event triggering natriuretic peptide release into the tissues and the time the sample is obtained or analyzed; the elapsed time between sample acquisition and the time the sample is analyzed; the type of tissue sample at issue; the storage conditions; the quantity of proteolytic enzymes present; etc., such fragments may be used when both designing an assay for one or more natriuretic peptides, and when performing such an assay, in order to provide an accurate prognostic or diagnostic result.

### FIELD OF THE INVENTION

The present invention relates to the identification and use of polypeptides that are derived from biological active peptides, the peptides generated when the biological peptide is generated and the precursors of the aforementioned peptides.

### BACKGROUND OF THE INVENTION

The following discussion of the background of the invention is merely provided to aid the reader in understanding the invention and is not admitted to describe or constitute prior art to the present invention.

Natriuretic peptides are a group of naturally occurring substances that act in the body to oppose the activity of the renin-angiotensin system. There are three major natriuretic peptides: atrial natriuretic peptide (ANP), which is synthesized in the atria; brain-type natriuretic peptide (BNP), which is synthesized in the ventricles; and C-type natriuretic peptide (CNP), which is synthesized in the brain.

Mature A-type natriuretic peptide (ANP) (also referred to as atrial natriuretic peptide) is a 28 amino acid peptide that is synthesized, stored, and released by atrial myocytes in response to atrial distension, angiotensin II stimulation, endothelin, and sympathetic stimulation (beta-adrenoceptor mediated). Mature ANP is synthesized as a precursor molecule (pro-ANP) that is converted to an active form by proteolytic cleavage. In addition to atrial natriuretic peptide (ANP₉₉₋₁₂₆) itself, linear peptide fragments from its N-terminal prohormone segment have also been reported to have biological activity.

Mature B-type natriuretic peptide (BNP) (also called brain-type natriuretic peptide) is a 32 amino acid, 4 kDa peptide that is involved in the natriuresis system to regulate blood pressure and fluid balance (Bonow, R. O., Circulation 93:1946-1950, 1996). The precursor to BNP is synthesized as a 108-amino acid molecule, referred to herein as “pro-BNP” that is proteolytically processed into a 76-amino acid N-terminal peptide (amino acids 1-76), referred to as “NT pro BNP” and the 32-amino acid mature hormone, referred to as BNP or BNP 32 (amino acids 77-108). It has been suggested that each of these species—NT pro-BNP, BNP-32, and the pre-pro-BNP—can circulate in human plasma (Tateyama et al., Biochem. Biophys. Res. Commun. 185:760-7, 1992; Hunt et al., Biochem. Biophys. Res. Commun. 214:1175-83, 1995).

Mature C-type natriuretic peptide (CNP) a 22-amino acid peptide that is the primary active natriuretic peptide in the human brain; CNP is also considered to be an endothelium-derived relaxant factor, which acts in the same way as nitric oxide (NO) (Davidson et al., Circulation 93:1155-9, 1996). CNP is structurally related to A-type natriuretic peptide (ANP) and B-type natriuretic peptide (BNP); however, while ANP and BNP are synthesized predominantly in the myocardium, CNP is synthesized in the vascular endothelium as a precursor (pro-CNP) (Prickett et al, Biochem. Biophys. Res. Commun. 286:513-7, 2001). CNP is thought to possess vasodilator effects on both arteries and veins and has been reported to act mainly on the vein by increasing the intracellular cGMP concentration in vascular smooth muscle cells.

ANP and BNP are released in response to atrial and ventricular stretch, respectively, and will cause vasorelaxation, inhibition of aldosterone secretion in the adrenal cortex, and inhibition of renin secretion in the kidney. Both ANP and BNP will cause natriuresis and a reduction in intravascular volume, effects amplified by the antagonism of antidiuretic hormone (ADH). The physiologic effects of CNP differ from those of ANP and BNP; CNP has a hypotensive effect, but no significant diuretic or natriuretic actions. Increased blood levels of natriuretic peptides have been found in certain disease states, suggesting a role in the pathophysiology of those diseases, including stroke, congestive heart failure (CHF), cardiac ischemia, systemic hypertension, and acute myocardial infarction. See, e.g., WO 02/089657; WO 02/083913; and WO 03/016910, each of which is hereby incorporated in its entirety, including all tables, figures, and claims.

The natriuretic peptides, alone, collectively, and/or together with additional proteins, can also serve as disease markers and indicators of prognosis in various cardiovascular conditions. For example, BNP, which is synthesized in the cardiac ventricles and correlates with left ventricular pressure, amount of dyspnea, and the state of neurohormonal modulation, makes this peptide the first potential marker for heart failure. Measurement of plasma BNP concentration is evolving as a very efficient and cost effective mass screening technique for identifying patients with various cardiac abnormalities regardless of etiology and degree of LV systolic dysfunction that can potentially develop into obvious heart failure and carry a high risk of a cardiovascular event. Finding a simple blood test that would aid in the diagnosis and management of patients with CHF clearly would have a favorable impact on the staggering costs associated with the disease.

Removal of the natriuretic peptides from the circulation is affected mainly by binding to clearance receptors and enzymatic degradation in the circulation. See, e.g., Cho et al., Heart Dis. 1: 305-28, 1999; Smith et al., J. Endocrinol. 167: 239-46, 2000. Additionally, human pro-BNP is reported to be processed in serum such that circulating pre-pro-BNP is unlikely to be the intact 108 amino acid form. Hunt et al., Peptides 18: 1475-81, 1997. But some confusion over the stability of the natriuretic peptides, particularly in blood-derived samples (e.g., serum, plasma, whole blood) has been reported. For example, while Norman et al. (Biochem. Biophys. Res. Commun. 28: 175: 22-30, 1991) report that neutral endopeptidase can cleave human BNP between residues 2 and 3, between residues 4 and 5, and between residues 17 and 18, Smith et al. (J. Endocrinol. 167: 239-46, 2000) report that human BNP is not significantly degraded by purified neutral endopeptidase. Similarly, Shimizu et al. (Clin. Chem. Acta 305: 181-6, 2001), Gobinet-Georges et al. (Clin. Chem. Lab. Med. 38: 519-23, 2000) and Murdoch et al. (Heart 78: 594-7, 1997) report that BNP is stable in certain blood-derived samples or when blood is collected under certain conditions. A more recent report by Shimizu et al. (Clin. Chem. Acta 316: 129-35, 2002) indicates that 94% of BNP in whole blood was a digested form in which 2 amino terminal residues had been removed; and that BNP in plasma was degraded to a number of unidentified forms.

### BRIEF SUMMARY OF THE INVENTION

The present invention relates to the identification and use of polypeptides that are derived from natriuretic peptides and their precursors. Using BNP as an example, the present invention describes a number of degradation products of natriuretic peptides produced in biological samples, most preferably blood-derived samples. Because production of such fragments is an ongoing process that may be a function of, inter alia, the elapsed time between onset of an event triggering natriuretic peptide release into the tissues and the time the sample is obtained or analyzed; the elapsed time between sample acquisition and the time the sample is analyzed; the type of tissue sample at issue; the storage conditions; the quantity of proteolytic enzymes present; etc., it may be necessary to consider this degradation when both designing an assay for one or more natriuretic peptides, and when performing such an assay, in order to provide an accurate prognostic or diagnostic result.

As described hereinafter, in one embodiment an assay may be conducted using an antibody or antibody cocktail formulated to detect a plurality of natriuretic peptide (e.g., BNP) fragments as defined herein. The presence or amount of this plurality of fragments may provide a more accurate prognostic or diagnostic result than simply measuring the mature natriuretic peptide (or natriuretic peptide precursor) itself. For example, antibodies that detect only the mature natriuretic peptide, but that are not able to detect degradation fragments, may provide an aberrantly low assay result (e.g., indicating that no BNP or low BNP concentrations are present in the sample, when the BNP was present, but has been degraded).

In an alternative embodiment, individual antibodies that distinguish amongst a plurality of natriuretic peptide (e.g., BNP) fragments may be individually employed to separately detect the presence or amount of different fragments. The results of this individual detection may provide a more accurate prognostic or diagnostic result than detecting the plurality of fragments in a single assay. For example, different weighting factors may be applied to the various fragment measurements to provide a more accurate estimate of the amount of natriuretic peptide originally present in the sample. Additionally, the relative amounts of the various fragments may be used to estimate the length of time since the onset of an event since, as discussed above, production of such fragments may be a function of, inter alia, the elapsed time between onset of an event triggering natriuretic peptide release into the tissues and the time the sample is obtained or analyzed.

In yet another alternative, a sample may be mixed with one or more compounds that inhibit the production of natriuretic peptide (e.g., BNP) fragments. In such embodiments, one or more proteolytic inhibitors and/or chelators may be added to a biological sample to prevent degradation of the natriuretic peptide(s) fragments that may not be accurately detected by an assay.

The methods and compositions described herein can meet the need in the art for rapid, sensitive and specific diagnostic assay to be used in the diagnosis and differentiation of various cardiovascular diseases, including stroke, congestive heart failure (CHF), cardiac ischemia, systemic hypertension, and/or acute myocardial infarction. Moreover, the methods and compositions of the present invention can also be used to facilitate the treatment of patients and the development of additional diagnostic and/or prognostic indicators and indicator panels.

In a first aspect, the present invention relates to one or more purified, and preferably substantially purified, natriuretic peptide fragments other than mature ANP, BNP, and CNP, their precursor molecules, and the fragments generated by cleavage of the precursor molecules into the mature ANP, BNP, and CNP peptides. As discussed above, the present invention is described using human BNP as an exemplary source for such fragments. Human BNP is derived by proteolysis of a 108 amino acid precursor molecule, referred to hereinafter as BNP₁₋₁₀₈. Mature BNP, or “the BNP natriuretic peptide,” is a 32 amino acid molecule representing amino acids 77-108 of this precursor, and is referred to hereinafter as BNP₇₇₋₁₀₈. The remaining residues 1-76 are referred to hereinafter as BNP₁₋₇₆.

The sequence of the 108 amino acid BNP precursor pro-BNP (BNP₁₋₁₀₈) is as follows, with mature BNP (BNP₇₇₋₁₀₈) underlined:

| | | |
|---|---|---|
| HPLGSPGSAS DLETSGLQEQ RNHLQGKLSE LQVEQTSLEP LQESPRPTGV | 50 | (SEQ ID NO: 1) |
| WKSREVATEG IRGHRKMVLY TLRAPRSPKM VQGSGCFGRK MDRISSSSGL | 100 | |
| GCKVLRRH. 108 | | |

BNP₁₋₁₀₈ is synthesized as a larger precursor pre-pro-BNP having the following sequence (with the “pre” sequence shown in bold):

| | | |
|---|---|---|
| MDPQTAPSRA LLLLLFLHLA FLGGRSHPLG SPGSASDLET SGLQEQRNHL | 50 | (SEQ ID NO: 2) |
| QGKLSELQVE QTSLEPLQES PRPTGVWKSR EVATEGIRGH RKMVLYTLRA | 100 | |
| PRSPKMVQGS GCFGRKMDRI SSSSGLGCKV LRRH. 134 | | |

The sequence of the 126 amino acid ANP precursor pro-ANP (ANP₁₋₁₂₆) is as follows, with mature ANP (ANP₉₉₋₁₂₆) underlined:

| | | |
|---|---|---|
| NPMYNAVSNA DLMDFKNLLD HLEEKMPLED EVVPPQVLSD PNEEAGAALS | 50 | (SEQ ID NO: 3) |
| PLPEVPPWTG EVSPAQRDGG ALGRGPWDSS DRSALLKSKL RALLTAPRSL | 100 | |
| RRSSCFGGRM DRIGAQSGLG CNSFRY. 126 | | |

ANP₁₋₁₂₆ is synthesized as a larger precursor pre-pro-ANP having the following sequence (with the “pre” sequence shown in bold):

| | | |
|---|---|---|
| MSSFSTTTVS FLLLLAFQLL GQTRANPMYN AVSNADLMDF KNLLDHLEEK | 50 | (SEQ ID NO: 4) |
| MPLEDEVVPP QVLSDPNEEA GAALSPLPEV PPWTGEVSPA QRDGGALGRG | 100 | |
| PWDSSDRSAL LKSKLRALLT APRSLRRSSC FGGRMDRIGA QSGLGCNSFR | 150 | |
| Y. 151 | | |

The sequence of the 126 amino acid CNP precursor pro-CNP (CNP₁₋₁₂₆) is as follows, with the mature CNP forms CNP-53 (CNP₇₄₋₁₂₆) in italics, and CNP-22 (CNP₁₀₅₋₁₂₆) underlined:

| | | |
|---|---|---|
| MHLSQLLACA LLLTLLSLRP SEAKPGAPPK VPRTPPAEEL AEPQAAGGGQ | 50 | (SEQ ID NO: 5) |
| KKGDKAPGGG GANLKGDRSR LLRDLRVDTK SRAAWARLLQ EHPNARKYKG | 100 | |
| ANKKGLSKGC FGLKLDRIGS MSGLGC. 126 | | |

In various embodiments, the present invention relates to any purified, and preferably substantially purified, BNP-related polypeptide(s) other than pre-pro-BNP, BNP₁₋₁₀₈, BNP₁₋₇₆, and BNP₇₇₋₁₀₈. In preferred embodiments, the present invention relates to one or more substantially purified BNP-related polypeptides selected from the group consisting of BNP₇₇₋₁₀₆, BNP₇₉₋₁₀₆, BNP₇₆₋₁₀₇, BNP₆₉₋₁₀₈, BNP₇₉₋₁₀₈, BNP₈₀₋₁₀₈, BNP₈₁₋₁₀₈, BNP₈₃₋₁₀₈, BNP₃₉₋₈₆, BNP₅₃₋₈₅, BNP₆₆₋₉₈, BNP₃₀₋₁₀₃, BNP₁₁₋₁₀₇, BNP₉₋₁₀₆, and BNP₃₋₁₀₈. In preferred embodiments, BNP₁₋₁₀₈, BNP₇₉₋₁₀₈, BNP₈₀₋₁₀₈, BNP₈₆₋₁₀₈, BNP₇₇₋₁₀₇, BNP₇₇₋₁₀₆, BNP₇₇₋₁₀₃, BNP₁₋₁₃, and BNP₆₂₋₇₆ are excluded in their individually purified forms.

In addition, natriuretic peptide fragments, including BNP fragments, may comprise one or more oxidizable methionines, the oxidation of which to methionine sulfoxide or methionine sulfone. Changes in the oxidation state of one or more methionines may alter the ability of assays to detect such fragments. Thus, in addition to the reduced forms of the substantially purified natriuretic peptide fragments discussed above, the present invention also relates to one or more purified, and preferably substantially purified, natriuretic peptide fragments other than mature ANP, BNP, and CNP, their precursor molecules, and the fragments generated by cleavage of the precursor molecules into the mature ANP, BNP, and CNP peptides, in which one or more methionines are oxidized. Preferred are one or more substantially purified BNP-related polypeptides selected from the group consisting of BNP₇₇₋₁₀₆, BNP₇₉₋₁₀₆, BNP₇₆₋₁₀₇, BNP₆₉₋₁₀₈, BNP₇₇₋₁₀₈, BNP₇₉₋₁₀₈, BNP₈₀₋₁₀₈, BNP₈₁₋₁₀₈, BNP₈₃₋₁₀₈, BNP₃₉₋₈₆, BNP₅₃₋₈₅, BNP₆₆₋₉₈, BNP₃₀₋₁₀₃, BNP₁₁₋₁₀₇, BNP₉₋₁₀₆, and BNP₃₋₁₀₈ in which one or more methionines are oxidized. The presence or absence of natriuretic peptide fragments in which one or more of these peptides may be measured by immunoassay, mass spectrometry, high pressure liquid chromatography and gas chromatorgraphy, as described hereinafter.

The term “fragment” as used herein refers to a polypeptide that comprises at least six contiguous amino acids of a polypeptide from which the fragment is derived. Thus, a fragment of BNP₁₋₁₀₈ (pro-BNP) refers to a polypeptide that comprises at least six contiguous amino acids of BNP₁₋₁₀₈; a fragment of mature BNP refers to a polypeptide that comprises at least six contiguous amino acids of BNP₇₇₋₁₀₈; a fragment of the polypeptide generated by cleavage of pro-BNP into mature BNP refers to a polypeptide that comprises at least six contiguous amino acids of BNP₁₋₇₆. Similarly, a fragment of ANP₁₋₁₂₆ (pro-ANP) refers to a polypeptide that comprises at least six contiguous amino acids of ANP₁₋₁₂₆; a fragment of mature ANP refers to a polypeptide that comprises at least six contiguous amino acids of ANP₉₉₋₁₂₆; a fragment of the polypeptide generated by cleavage of pro-ANP into mature ANP refers to a polypeptide that comprises at least six contiguous amino acids of BNP₁₋₉₈; and a fragment of CNP₁₋₁₂₆ (pro-CNP) refers to a polypeptide that comprises at least six contiguous amino acids of CNP₁₋₁₂₆; a fragment of mature CNP refers to a polypeptide that comprises at least six contiguous amino acids of CNP₇₄₋₁₂₆ or CNP₁₀₅₋₁₂₆; a fragment of the polypeptide generated by cleavage of pro-CNP into mature CNP refers to a polypeptide that comprises at least six contiguous amino acids of CNP₁₋₇₃ or CNP₁₋₁₀₄. In preferred embodiments, a fragment refers to a polypeptide that comprises at least 10 contiguous amino acids of a polypeptide from which the fragment is derived; at least 15 contiguous amino acids of a polypeptide from which the fragment is derived; or at least 20 contiguous amino acids of a polypeptide from which the fragment is derived.

The term “natriuretic peptide fragment” as used herein refers to a fragment, as described above, of any natriuretic peptide selected from the group consisting of mature ANP, BNP, or CNP, the biosynthetic precursors pre-pro-ANP, pre-pro-BNP, pre-pro-CNP, pro-ANP, pro-BNP, or pro-CNP, or the polypeptide remaining after removal of mature ANP, BNP, or CNP from the pro-form of the peptide.

Most preferably, a fragment is “naturally present” in a biological sample (e.g., a blood, serum or plasma sample, and most preferably human blood, serum, or plasma). By this is meant that the fragment may be obtained from an unsupplemented biological sample obtained from a human or animal. “Unsupplemented” refers to a sample in which the fragment or its precursor has not been exogenously added once the sample is obtained. Examples of fragments naturally present in blood, serum or plasma are described hereinafter. Other preferred fragments are said to be “generated from” blood, serum or plasma if the fragment is present as a result of supplementing such a sample with pro-ANP, pro-BNP, pro-CNP, and/or a fragment thereof, and allowing endogenous factors (e.g., proteases) in the sample to generate additional fragments. Examples of fragments generated from human blood, serum or plasma are also described hereinafter. A fragment is “present” in blood, serum or plasma if the fragment is either naturally present or generated from such a sample.

As used herein, the term “purified” in reference to polypeptides does not require absolute purity. Instead, it represents an indication that the polypeptide(s) of interest is (are) in a discrete environment in which abundance (on a mass basis) relative to other proteins is greater than in a biological sample. By “discrete environment” is meant a single medium, such as a single solution, a single gel, a single precipitate, etc. Purified polypeptides may be obtained by a number of methods including, for example, laboratory synthesis, chromatography, preparative electrophoresis, centrifugation, precipitation, affinity purification, etc. One or more “purified” polypeptides of interest are preferably at least 10% of the protein content of the discrete environment. One or more “substantially purified” polypeptides are at least 50% of the protein content of the discrete environment, more preferably at least 75% of the protein content of the discrete environment, and most preferably at least 95% of the protein content of the discrete environment. Protein content is determined using a modification of the method of Lowry et al., J. Biol. Chem. 193: 265, 1951, described by Hartree, Anal Biochem 48: 422-427 (1972), using bovine serum albumin as a protein standard.

In related aspects, the purified natriuretic peptide fragments of the present invention may be employed in methods to generate antibodies that recognize one or a group of fragments. In various embodiments, a polypeptide may be selected that comprises a sequence that is common to a number of natriuretic peptide fragments, and used to generate antibodies that recognize this common sequence; such antibodies would recognize each of the fragments in which the sequence is in common and expressed such that binding is sterically possible. In alternative embodiments, a fragment may be selected that comprises a sequence that is distinctive to a specific fragment or set of fragments, and used to generate antibodies that recognize only that particular fragment or set of fragments. Such an antibody is said to “distinguish” the selected fragments from those fragments that are unrecognized by the antibody. Thus, the present invention also relates to antibodies selected to bind one or more preselected natriuretic peptide fragments, and methods for their generation and selection.

In various embodiments, the present invention relates to antibodies selected to bind to a plurality of BNP-related polypeptides selected from the group consisting of BNP₁₋₁₀₈, BNP₁₋₇₆, BNP₇₇₋₁₀₈, BNP₇₇₋₁₀₆, BNP₇₉₋₁₀₆, BNP₇₆₋₁₀₇, BNP₆₉₋₁₀₈, BNP₇₉₋₁₀₈, BNP₈₀₋₁₀₈, BNP₈₁₋₁₀₈, BNP₈₃₋₁₀₈, BNP₃₉₋₈₆, BNP₅₃₋₈₅, BNP₆₆₋₉₈, BNP₃₀₋₁₀₃, BNP₁₁₋₁₀₇, BNP₉₋₁₀₆, and BNP₃₋₁₀₈. The present invention also relates to methods for the selection of such antibodies. Preferably, such antibodies are selected to bind to a plurality of BNP peptides generated from BNP₇₇₋₁₀₈, more preferably to bind a plurality of BNP₇₇₋₁₀₈, BNP₇₇₋₁₀₆, BNP₇₉₋₁₀₆, BNP₇₆₋₁₀₇, BNP₇₉₋₁₀₈, BNP₈₀₋₁₀₈, BNP₈₁₋₁₀₈, BNP₈₃₋₁₀₈, and most preferably to each of BNP₇₇₋₁₀₈, BNP₇₇₋₁₀₆, BNP₇₉₋₁₀₆, BNP₇₆₋₁₀₇, BNP₇₉₋₁₀₈, BNP₈₀₋₁₀₈, BNP₈₁₋₁₀₈, BNP₈₃₋₁₀₈. In other preferred embodiments, antibodies are also selected to bind to BNP-related polypeptides regardless of methionine oxidation state.

In various embodiments, the present invention relates to antibodies selected to specifically bind to a plurality of BNP-related polypeptides selected from the group consisting of BNP₁₋₁₀₈, BNP₁₋₇₆, BNP₇₇₋₁₀₈, BNP₇₇₋₁₀₆, BNP₇₉₋₁₀₆, BNP₇₆₋₁₀₇, BNP₆₉₋₁₀₈, BNP₇₉₋₁₀₈, BNP₈₀₋₁₀₈, BNP₈₁₋₁₀₈, BNP₈₃₋₁₀₈, BNP₃₉₋₈₆, BNP₅₃₋₈₅, BNP₆₆₋₉₈, BNP₃₀₋₁₀₃, BNP₁₁₋₁₀₇, BNP₉₋₁₀₆, and BNP₃₋₁₀₈. The present invention also relates to methods for the selection of such antibodies. Preferably, such antibodies are selected to bind specifically to a plurality of BNP peptides generated from BNP77-108, more preferably to bind a plurality of BNP₇₇₋₁₀₈, BNP₇₇₋₁₀₆, BNP₇₉₋₁₀₆, BNP₇₆₋₁₀₇, BNP₇₉₋₁₀₈, BNP₈₀₋₁₀₈, BNP₈₁₋₁₀₈, BNP₈₃₋₁₀₈, and most preferably to each of BNP₇₇₋₁₀₈, BNP₇₇₋₁₀₆, BNP₇₉₋₁₀₆, BNP₇₆₋₁₀₇, BNP₇₉₋₁₀₈, BNP₈₀₋₁₀₈, BNP₈₁₋₁₀₈, BNP₈₃₋₁₀₈. In other preferred embodiments, antibodies are also selected to bind specifically to BNP-related polypeptides regardless of methionine oxidation state.

In various alternative embodiments, the present invention relates to antibodies selected to distinguish between a first group comprising one or more BNP-related polypeptides selected from the group consisting of BNP₁₋₁₀₈, BNP₁₋₇₆, BNP₇₇₋₁₀₈, BNP₇₇₋₁₀₆, BNP₇₉₋₁₀₆, BNP₇₆₋₁₀₇, BNP₆₉₋₁₀₈, BNP₇₉₋₁₀₈, BNP₈₀₋₁₀₈, BNP₈₁₋₁₀₈, BNP₈₃₋₁₀₈, BNP₃₉₋₈₆, BNP₅₃₋₈₅, BNP₆₆₋₉₈, BNP₃₀₋₁₀₃, BNP₁₁₋₁₀₇, BNP₉₋₁₀₆, and BNP₃₋₁₀₈, and a second group comprising one or more different BNP-related polypeptides selected from the group consisting of BNP₁₋₁₀₈, BNP₁₋₇₆, BNP₇₇₋₁₀₈, BNP₇₇₋₁₀₆, BNP₇₉₋₁₀₆, BNP₇₆₋₁₀₇, BNP₆₉₋₁₀₈, BNP₇₉₋₁₀₈, BNP₈₀₋₁₀₈, BNP₈₁₋₁₀₈, BNP₈₃₋₁₀₈, BNP₃₉₋₈₆, BNP₅₃₋₈₅, BNP₆₆₋₉₈, BNP₃₀₋₁₀₃, BNP₁₁₋₁₀₇, BNP₉₋₁₀₆, and BNP₃₋₁₀₈. The present invention also relates to methods for the selection of such antibodies. Preferably, members of the first and/or second groups comprise BNP peptides generated from BNP₇₇₋₁₀₈, and most preferably members of the first and/or second groups comprise BNP₇₇₋₁₀₈, BNP₇₇₋₁₀₆, BNP₇₉₋₁₀₆, BNP₇₆₋₁₀₇, BNP₇₉₋₁₀₈, BNP₈₀₋₁₀₈, BNP₈₁₋₁₀₈, BNP₈₃₋₁₀₈. In other preferred embodiments, antibodies are also selected to distinguish BNP-related polypeptides on the basis of a methionine oxidation state.

The term “antibody” as used herein refers to a peptide or polypeptide derived from, modeled after or substantially encoded by an immunoglobulin gene or immunoglobulin genes, or fragments thereof, capable of specifically binding an antigen or epitope. See, e.g. Fundamental Immunology, 3^{rd} Edition, W. E. Paul, ed., Raven Press, N.Y. (1993); Wilson (1994) J. Immunol. Methods 175:267-273; Yarmush (1992) J. Biochem. Biophys. Methods 25:85-97. The term antibody includes antigen-binding portions, i.e., “antigen binding sites,” (e.g., fragments, subsequences, complementarity determining regions (CDRs)) that retain capacity to bind antigen, including (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Single chain antibodies, monoclonal antibodies, polyclonal antibodies, and antibodies obtained by molecular biological techniques (e.g., by phage display methods) are also included by reference in the term “antibody.” Preferred antibodies are “Omniclonal” antibodies. By this is meant a mixture of different antibody molecules selected from a phage display library, where each antibody specifically binds to a target antigen with a minimum affinity of 10⁹ M⁻¹ to 10¹⁰ M⁻¹.

The term “specifically binds” is not intended to indicate that an antibody binds exclusively to its intended target. Rather, an antibody specifically binds if its affinity for its intended target is about 2-fold greater when compared to its affinity for a non-target molecule. Preferably the affinity of the antibody will be at least about five fold, preferably 10 fold, more preferably 25-fold, even more preferably 50-fold, and most preferably 100-fold or more, greater for a target molecule than its affinity for a non-target molecule. In preferred embodiments, Specific binding between an antibody or other binding agent and an antigen means a binding affinity of at least 10.sup.6 M.sup.-1. Preferred antibodies bind with affinities of at least about 10⁷ M⁻¹, and preferably 10⁸ M⁻¹ to 10⁹ M⁻¹ or 10¹⁰ M⁻¹.

The term “plurality” as used herein in reference to natriuretic peptide fragments and BNP-related polypeptides refers to 2 or more molecular species that differ in amino acid sequence.

The skilled artisan will understand that individual antibodies (e.g., obtained by phage display or monoclonal antibody technology) may be obtained that bind to a plurality of fragments having a common epitope to which the antibody may bind. In the alternative, individual antibodies may be pooled to provide the desired spectrum of binding affinities. The term “antibody” as used herein may refer to both a composition in which each antibody molecule present is identical (referred to specifically as an “individual antibody”), or a composition in which antibody molecules present may differ (e.g., in a pooled or polyclonal composition).

In various embodiments, antibodies are selected, based not upon a particular affinity for one or more natriuretic peptide fragments, but instead based upon a signal that is obtainable in a binding assay such as an immunoassay. The skilled artisan will recognize that various binding assay formats are known in the art, and that it is often the use of antibodies to formulate an appropriate assay that is more important than a particular affinity of an antibody for one or more target molecules. For example, competitive binding assays may comprise a receptor (e.g., an antibody) bound to a solid surface. An analyte of interest in a test sample competes for binding with a labeled molecule that also binds to the receptor. The amount of labeled molecule bound to the receptor (and hence assay signal) is inversely proportional to the amount of analyte of interest in the test sample. In this case, a single antibody attached to the solid phase is used. Alternatively, in a sandwich immunoassay, a first antibody, typically bound to a solid surface, and a second antibody, typically conjugated to a detectable label, each bind to an analyte of interest in a test sample. The amount of labeled molecule bound to the receptor (and hence assay signal) is directly proportional to the amount of analyte of interest in the test sample.

Thus, in another aspect, the presence or amount of the various natriuretic peptide fragments present in a sample are determined. Such an analysis is preferably performed in an immunoassay using the antibodies of the present invention, although other methods are well known to those skilled in the art (for example, the use of biosensors, or the use of natural receptors for natriuretic peptides that are known in the art). Any suitable immunoassay may be utilized, for example, enzyme-linked immunoassays (ELISA), radioimmunoassays (RIAs), competitive binding assays, sandwich immunoassaysm and the like. Specific immunological binding of the antibody to the one or more natriuretic peptide fragments can be detected directly or indirectly. Direct labels include fluorescent or luminescent tags, metals, dyes, radionuclides, and the like, attached to the antibody. Indirect labels include various enzymes well known in the art, such as alkaline phosphatase, horseradish peroxidase and the like. Antibodies attached to a second molecule, such as a detectable label, are referred to herein as “antibody conjugates.” The skilled artisan will also understand that natural receptors for the natriuretic peptides exist, and that these receptors may also be used in a manner akin to antibodies in providing binding assays.

In various embodiments, the present invention relates to immunoassays configured to provide a single signal that relates to the presence or amount of a plurality of BNP-related polypeptides selected from the group consisting of BNP₁₋₁₀₈, BNP₁₋₇₆, BNP₇₇₋₁₀₈, BNP₇₇₋₁₀₆, BNP₇₉₋₁₀₆, BNP₇₆₋₁₀₇, BNP₆₉₋₁₀₈, BNP₇₉₋₁₀₈, BNP₈₀₋₁₀₈, BNP₈₁₋₁₀₈, BNP₈₃₋₁₀₈, BNP₃₉₋₈₆, BNP₅₃₋₈₅, BNP₆₆₋₉₈, BNP₃₀₋₁₀₃, BNP₁₁₋₁₀₇, BNP₉₋₁₀₆, and BNP₃₋₁₀₈. Preferably, such immunoassays configured to provide a single signal that is related to the presence or amount of a plurality of BNP peptides generated from BNP77-108, more preferably to a plurality of BNP₇₇₋₁₀₈, BNP₇₇₋₁₀₆, BNP₇₉₋₁₀₆, BNP₇₆₋₁₀₇, BNP₇₉₋₁₀₈, BNP₈₀₋₁₀₈, BNP₈₁₋₁₀₈, BNP₈₃₋₁₀₈, and most preferably to each of BNP₇₇₋₁₀₈, BNP₇₇₋₁₀₆, BNP₇₉₋₁₀₆, BNP₇₆₋₁₀₇, BNP₇₉₋₁₀₈, BNP₈₀₋₁₀₈, BNP₈₋₁₀₈, BNP₈₃₋₁₀₈. In other preferred embodiments, immunoassays are also configured to provide a single signal that relates to the presence or amount of BNP-related polypeptides regardless of methionine oxidation state.

In preferred embodiments, an immunoassay provides a signal that is within a factor of 5, and most preferably within a factor of two, from an equal number of molecules of a plurality of natriuretic peptide fragments, and most preferably a plurality of the foregoing BNP-related polypeptides.

In various alternative embodiments, the present invention relates to immunoassays configured to provide a signal that distinguishes between a first group comprising one or more BNP-related polypeptides selected from the group consisting of BNP₁₋₁₀₈, BNP₁₋₇₆, BNP₇₇₋₁₀₈, BNP₇₇₋₁₀₆, BNP₇₉₋₁₀₆, BNP₇₆₋₁₀₇, BNP₆₉₋₁₀₈, BNP₇₉₋₁₀₈, BNP₈₀₋₁₀₈, BNP₈₁₋₁₀₈, BNP₈₃₋₁₀₈, BNP₃₉₋₈₆, BNP₅₃₋₈₅, BNP₆₆₋₉₈, BNP₃₀₋₁₀₃, BNP₁₁₋₁₀₇, BNP₉₋₁₀₆, and BNP₃₋₁₀₈, and a second group comprising one or more different BNP-related polypeptides selected from the group consisting of BNP₁₋₁₀₈, BNP₇₇₋₁₀₆, BNP₇₇₋₁₀₈, BNP₇₇₋₁₀₆, BNP₇₉₋₁₀₆, BNP₇₆₋₁₀₇, BNP₆₉₋₁₀₈, BNP₇₉₋₁₀₈, BNP₈₀₋₁₀₈, BNP₈₁₋₁₀₈, BNP₈₃₋₁₀₈, BNP₃₉₋₈₆, BNP₅₃₋₈₅, BNP₆₆₋₉₈, BNP₃₀₋₁₀₃, BNP₁₁₋₁₀₇, BNP₋₁₀₆, and BNP₃₋₁₀₈. Preferably, members of the first and/or second groups comprise BNP peptides generated from BNP₇₇₋₁₀₈, and most preferably members of the first and/or second groups comprise BNP₇₇₋₁₀₈, BNP₇₇₋₁₀₆, BNP₇₉₋₁₀₆, BNP₇₆₋₁₀₇, BNP₇₉₋₁₀₈, BNP₈₀₋₁₀₈, BNP₈₁₋₁₀₈, BNP₈₃₋₁₀₈. In other preferred embodiments, immunoassays are also configured to distinguish BNP-related polypeptides depending upon methionine oxidation state.

An immunoassay is said to “distinguish” between a first group of polypeptides and a second group of polypeptides if the immunoassay provides a signal related to binding of the first group of polypeptides that is at least a factor of 10 greater than a signal obtained from an equal number of molecules of the second group of polypeptides under the same assay conditions. More preferably, the signal is at least a factor of 20 greater, even more preferably at least a factor of 50 greater, and most preferably at least a factor of 100 greater or more.

An antibody is said to “distinguish” between a first group of polypeptides and a second group of polypeptides if its affinity for the members of the first group of polypeptides is about 2-fold greater when compared to its affinity for members of the second group. Preferably the affinity of the antibody will be at least about five fold, preferably 10 fold, more preferably 25-fold, even more preferably 50-fold, and most preferably 100-fold or more, greater for members of the first group of polypeptides than its affinity for members of the second group.

A signal from an immunoassay is said to “depend upon binding to an antibody” if the antibody participates in formation of a complex necessary to generate the signal. For example, in a sandwich immunoassay formulated using a solid phase antibody and a second antibody conjugate, each of which must bind to an analyte to form the sandwich, each of the solid phase antibody and second antibody participate in formation of the complex necessary to generate the signal. In a competitive immunoassay where a single antibody is used, and an analyte competes with an analyte conjugate for binding, the single antibody participates in formation of the complex necessary to generate the signal. The skilled artisan will understand that numerous additional immunoassay formulations may be provided.

Devices for performing the assays described herein preferably contain a plurality of discrete, independently addressable locations, or “diagnostic zones,” each of which is related to a particular peptide or set of peptides of interest. For example, each of a plurality of discrete zones may comprise a receptor (e.g., an antibody) for binding a different peptide. Alternatively, one or more zones may each comprise a receptor (e.g., an antibody) for binding a plurality of peptides. Following reaction of a sample with the devices, a signal is generated from the diagnostic zone(s), which may then be correlated to the presence or amount of the peptide of interest.

The term “discrete” as used herein refers to areas of a surface that are non-contiguous. That is, two areas are discrete from one another if a border that is not part of either area completely surrounds each of the two areas. The term “independently addressable” as used herein refers to discrete areas of a surface from which a specific signal may be obtained. One skilled in the art will appreciate that antibody zones can also be independent of each other, but can be in contact with each other on a surface.

The term “test sample” as used herein refers to a sample in which the presence or amount of one or more analytes of interest are unknown and to be determined in an assay, preferably an immunoassay. Preferably, a test sample is a bodily fluid obtained for the purpose of diagnosis, prognosis, or evaluation of a subject, such as a patient. In certain embodiments, such a sample may be obtained for the purpose of determining the outcome of an ongoing condition or the effect of a treatment regimen on a condition. Preferred test samples include blood, serum, plasma, cerebrospinal fluid, urine and saliva. In addition, one of skill in the art would realize that some test samples would be more readily analyzed following a fractionation or purification procedure, for example, separation of whole blood into serum or plasma components. Preferred samples may be obtained from bacteria, viruses and animals, such as dogs and cats. Particularly preferred samples are obtained from humans. By way of contrast, a “standard sample” refers to a sample in which the presence or amount of one or more analytes of interest are known prior to assay for the one or more analytes.

The term “disease sample” as used herein refers to a tissue sample obtained from a subject that has been determined to suffer from a given disease. Methods for clinical diagnosis are well known to those of skill in the art. See, e.g., Kelley's Textbook of Internal Medicine, 4^{th} Ed., Lippincott Williams & Wilkins, Philadelphia, Pa., 2000; The Merck Manual of Diagnosis and Therapy, 17^{th} Ed., Merck Research Laboratories, Whitehouse Station, N.J., 1999.

The skilled artisan will understand that the presence or amount of one or more natriuretic peptide fragments of interest may be related to the presence or absence of a disease, or the likelihood of a future adverse outcome related to a disease. However, the signal obtained from an assay need not be related to the presence or amount of one or more natriuretic peptide fragments; rather, the signal may be directly related to the presence or absence of a disease, or the likelihood of a future adverse outcome related to a disease. For example, a level of signal x may indicate that y pg/mL of a fragment is present in the sample. A table may then indicate that y pg/mL of that fragment indicates congestive heart failure. It may be equally valid to simply relate a level of signal x directly to congestive heart failure, without determining how much of the fragment is present. Such a signal is preferably obtained from an immunoassay using the antibodies of the present invention, although other methods are well known to those skilled in the art.

In another aspect, a test sample is contacted with one or more protease inhibitors to prevent degradation of natriuretic peptides and natriuretic peptide fragments contained in the sample. While this will not prevent degradation of peptides and peptide fragments occurring prior to sample collection (i.e., within the body), these methods can inhibit further degradation. Because the identity and amount of the various peptides and fragments may depend upon the elapsed time between onset of an event triggering peptide release into the tissues and the time the sample is obtained or analyzed, preventing further degradation may allow the sample to better reflect onset of the triggering event.

Suitable protease inhibitors and chelators for use in the present methods include, but are not limited to, phenylmethylsulfonyl fluoride (PMSF), diisopropylfluorophosphate (DFP), Pefabloc SC (4-(2-aminoethyl)-benzenesulfonyl fluoride), antipain, calpain inhibitors I and II, chymostatin, L-1-chloro-3-[4-tosylamido]-7-amino-2-heptanone (TLCK), soybean trypsin inhibitor, antithrombin III, aprotinin, 3,4-dichloroisocoumarin, 4-amidino-phenylmethylsulfonyl fluoride (APMSF), leupeptin, bestatin, E-64, EDTA, EGTA, hirudin, α-2-macroglobulin, pepstatin, phosphoramidon, and TIMP-2. Such supplemented test samples may be used in the assay methods described herein.

In another aspect, the presence or amount of the various natriuretic peptide fragments present in a sample are determined. Such an analysis is preferably performed in an immunoassay using the antibodies of the present invention, although other methods are well known to those skilled in the art (for example, the use of biosensors, or the use of natural receptors for natriuretic peptides that are known in the art). Any suitable immunoassay may be utilized, for example, enzyme-linked immunoassays (ELISA), radioimmunoassays (RIAs), competitive binding assays, sandwich immunoassaysm and the like. Specific immunological binding of the antibody to the one or more natriuretic peptide fragments can be detected directly or indirectly. Direct labels include fluorescent or luminescent tags, metals, dyes, radionuclides, and the like, attached to the antibody. Indirect labels include various enzymes well known in the art, such as alkaline phosphatase, horseradish peroxidase and the like. Antibodies attached to a second molecule, such as a detectable label, are referred to herein as “antibody conjugates.” The skilled artisan will also understand that natural receptors for the natriuretic peptides exist, and that these receptors may also be used in a manner akin to antibodies in providing binding assays.

In yet another aspect, the present invention relates to standard solutions comprising a known amount of one or more purified, and preferably substantially purified, natriuretic peptide fragments other than mature ANP, BNP, and CNP, their precursor molecules, and the fragments generated by cleavage of the precursor molecules into the mature ANP, BNP, and CNP peptides. Such standard solutions may find use as positive and/or negative control samples in the various assays described herein. In various embodiments, the present invention relates to any purified, and preferably substantially purified, BNP-related polypeptide(s) other than BNP₁₋₁₀₈, BNP₁₋₇₆, and BNP₇₇₋₁₀₈. In preferred embodiments, the present invention relates to one or more standard solutions comprising a known amount of one or more purified, and preferably substantially purified-related polypeptides selected from the group consisting of BNP₇₇₋₁₀₆, BNP₇₉₋₁₀₆, BNP₇₆₋₁₀₇, BNP₆₉₋₁₀₈, BNP₇₉₋₁₀₈, BNP₈₀₋₁₀₈, BNP₈₁₋₁₀₈, BNP₈₃₋₁₀₈, BNP₃₉₋₈₆, BNP₅₃₋₈₅, BNP₆₆₋₉₈, BNP₃₀₋₁₀₃, BNP₁₁₋₁₀₇, BNP₉₋₁₀₆, and BNP₃₋₁₀₈

In certain aspects, it may be advantageous to formulate such standard solutions using a composition that is substantially equivalent to the test sample; for example, the solution may comprise blood, serum, plasma, etc., as a solvent for the natriuretic peptide fragment(s) of interest. In such a case, it may also be advantageous to include one or more protease inhibitors or chelators in order to prevent degradation of the added natriuretic peptide fragment(s). Suitable protease inhibitors and chelators include, but are not limited to, phenylmethylsulfonyl fluoride (PMSF), diisopropylfluorophosphate (DFP), Pefabloc SC (4-(2-aminoethyl)-benzenesulfonyl fluoride), antipain, calpain inhibitors I and II, chymostatin, L-1-chloro-3-[4-tosylamido]-7-amino-2-heptanone (TLCK), soybean trypsin inhibitor, antithrombin III, aprotinin, 3,4-dichloroisocoumarin, 4-amidino-phenylmethylsulfonyl fluoride (APMSF), leupeptin, bestatin, E-64, EDTA, EGTA, hirudin, α-2-macroglobulin, pepstatin, phosphoramidon, and TIMP-2.

In another aspect, one or more antibodies, antibody conjugates, and/or standard solutions of the present invention may be provided as kits for determining the presence or amount of natriuretic peptide fragments. These kits preferably comprise devices and reagents for performing at least one assay as described herein on a test sample. Such kits preferably contain sufficient reagents to perform one or more such determinations, and/or Food and Drug Administration (FDA)-approved labeling.

In still another aspect, the invention relates to methods for determining a treatment regimen for use in a patient. The methods preferably comprise determining the presence or amount of one or more natriuretic peptide fragments other than mature ANP, BNP, and CNP, their precursor molecules, and the fragments generated by cleavage of the precursor molecules into the mature ANP, BNP, and CNP peptides, and relating this presence or amount to a disease or prognostic state. As discussed herein, diagnosis and differentiation of various cardiovascular and cerebrovascular diseases, including stroke, congestive heart failure (CHF), cardiac ischemia, systemic hypertension, and/or acute myocardial infarction may be related to ANP, BNP, and/or CNP levels. Once a diagnosis is obtained, a treatment regimen is selected to be consistent with that diagnosis.

In yet another aspect, the present invention relates to methods of identifying novel polypeptides present in biological samples, preferably blood, serum, or plasma samples, that are related to known polypeptides. In these methods, an antibody having an affinity for one or more known polypeptides (e.g., BNP) is used as an affinity probe for binding additional polypeptides that are sufficiently related in structure so as to share binding affinity to the antibody, but that are previously unpredicted as being present in the sample. The sequence of the polypeptide(s) is (are) then obtained by the methods described herein. Once obtained, the sequence may be used in the other aspects described herein; e.g., to select antibodies that can differentiate the known polypeptide(s) and the previously unknown polypeptides, again according to the methods described herein; to determine if the previously unknown polypeptides are useful as diagnostic or prognostic markers; and/or to provide standard solutions or isolated peptides.

The term “unpredicted polypeptides” as used herein refers to a polypeptide that, in the particular type of biological sample being analyzed, has not previously been demonstrated to be naturally present. A polypeptide is preferably unpredicted in a blood, serum, or plasma sample, and most preferably a human blood, serum, or plasma sample.

The term “determining the amino acid sequence” as used herein refers to methods by which the amino acid sequence of a particular polypeptide is obtained. Such methods may include direct sequencing (e.g., by Edman degradation); identification by mass spectrometry, which may comprise comparison of observed m/z to a predicted or known polypeptide sequence (see, e.g., Cagney and Emili, Nature Biotechnol. 20: 163-170 (2002)); peptide mapping; etc.

The summary of the invention described above is non-limiting and other features and advantages of the invention will be apparent from the following detailed description of the invention, and from the claims.

### DETAILED DESCRIPTION OF THE INVENTION

Use of Natriuretic Peptide Fragments as Prognostic and Diagnostic Markers

As noted above, increased blood levels of natriuretic peptides have been found in certain disease states, suggesting a role in the pathophysiology of those diseases, including stroke, congestive heart failure (CHF), cardiac ischemia, systemic hypertension, and acute myocardial infarction. See, e.g., WO 02/089657; WO 02/083913; WO 03/016910; Hunt et al., Biochem. Biophys. Res. Comm. 214:1175-83 (1995); Venugopal, J. Clin. Pharm. Ther. 26: 15-31, 2001; and Kalra et al., Circulation 107: 571-3, 2003; each of which is hereby incorporated in its entirety, including all tables, figures, and claims. The natriuretic peptides, alone, collectively, and/or together with additional proteins, can also serve as disease markers and indicators of prognosis in various cardiovascular conditions.

It has been reported that removal of natriuretic peptides from the circulation involves degradation pathways. Indeed, inhibitors of neutral endopeptidase, which cleaves natriuretic peptides under certain circumstances, have been suggested to hold promise in treatment of certain cardiovascular diseases. See, e.g., Trindade and Rouleau, Heart Fail. Monit. 2: 2-7, 2001. However, the measurement of the natriuretic peptides in clinical samples has focused generally upon measurement of the mature BNP, ANP, and/or CNP; their precursor molecules (i.e., pro-BNP, pro-ANP, and pro-CNP); and the fragments resulting from cleavage of the pro-form to provide the mature natriuretic peptides. The present invention describes for the first time a number of fragments produced by degradation of these molecules in biological samples. While described hereinafter mainly with reference to BNP-related fragments, the skilled artisan will understand that the general concepts described herein apply equally to ANP- and CNP-related fragments.

The failure to consider the degradation fragments that may be present in a clinical sample when measuring one or more of the natriuretic peptides may have serious consequences for the accuracy of any diagnostic or prognostic method. Consider for example a simple case, where a sandwich immunoassay is provided for BNP, and all of the BNP present has been degraded into two fragments, one of which contains the epitope corresponding to the solid phase antibody, the other of which contains the epitope corresponding to the antibody conjugate used for signal generation in the immunoassay. Because no BNP fragments present contain both epitopes, no signal will be obtained from the immunoassay, thus leading to the incorrect assumption that no BNP was originally present in the sample.

Similarly, another simple case may be considered. In a competitive assay, in which BNP present in solution competes with labeled BNP for binding to a solid phase antibody, consider that the solid phase is configured with a polyclonal antibody that would recognize both of the foregoing fragments. Each would bind to the antibody solid phase, and compete with the labeled BNP for binding. Such a situation may lead to the incorrect assumption that twice the BNP concentration actually present in the sample is detected.

As described herein, the situation may actually be much more complicated than these simple situations. In addition to BNP₁₋₁₀₈, BNP₁₋₇₆, and BNP₇₇₋₁₀₈. Which represent pro-BNP, the pro fragment, and mature BNP, the following degradation fragments have been identified in human serum or plasma: BNP₇₇₋₁₀₆, BNP₇₉₋₁₀₆, BNP₇₆₋₁₀₇, BNP₆₉₋₁₀₈, BNP₇₉₋₁₀₈, BNP₈₀₋₁₀₈, BNP₈₁₋₁₀₈, BNP₈₃₋₁₀₈, BNP₃₉₋₈₆, BNP₅₃₋₈₅, BNP₆₆₋₉₈, BNP₃₀₋₁₀₃, BNP₁₁₋₁₀₇, BNP₉₋₁₀₆, and BNP₃₋₁₀₈. Furthermore, methionine residues in fragments containing such amino acids may become oxidized, further complicating the degradation pattern. Failure to consider this degradation can result in an incorrect estimate of the amount of BNP present, with a concomitant error in diagnosis or prognosis.

Moreover, the failure to consider these fragments may also be discarding useful information for use in diagnosis or prognosis. As discussed above, production of such fragments is an ongoing process that may be a function of, inter alia, the elapsed time between onset of an event triggering natriuretic peptide release into the tissues and the time the sample is obtained or analyzed; the elapsed time between sample acquisition and the time the sample is analyzed; the type of tissue sample at issue; the storage conditions; the quantity of proteolytic enzymes present; etc. Determination of the relative pattern of degradation may be indicative of time of adverse event; the success (or lack thereof) in treatment with protease inhibitors; whether sample storage has been adequate, etc. Moreover, the individual fragments may also find use as markers in marker panels, with or without additional markers unrelated to natriuretic peptides. Additional unrelated markers include those in WO 02/089657; WO 02/083913; and WO 03/016910, each of which is hereby incorporated in their entirety, including all tables figured and claims.

The skilled artisan will understand that the methods described herein are applicable generally to polypeptides, and the analysis of the natriuretic peptides described in detail herein is merely exemplary. Other suitable polypeptides that may be the subject of similar analysis include angiotensin I, angiotensin II, vasopressin, calcitonin, calcitonin gene related peptide, urodilatin, urotensin II, free cardiac troponin I, free cardiac troponin T, cardiac troponin I in a complex comprising one or both of troponin T and troponin C, cardiac troponin T in a complex comprising one or both of troponin I and troponin C, total cardiac troponin I, total cardiac troponin T, pulmonary surfactant protein D, D-dimer, annexin V, enolase, creatine kinase, glycogen phosphorylase, heart-type fatty acid binding protein, phosphoglyceric acid mutase, S-100, S-100ao, plasmin-α2-antiplasmin complex, β-thromboglobulin, platelet factor 4, fibrinopeptide A, platelet-derived growth factor, prothrombin fragment 1+2, P-selectin, thrombin-antithrombin III complex, von Willebrand factor, tissue factor, thrombus precursor protein, human neutrophil elastase, inducible nitric oxide synthase, lysophosphatidic acid, malondialdehyde-modified low density lipoprotein, matrix metalloproteinase-1, matrix metalloproteinase-2, matrix metalloproteinase-3, matrix metalloproteinase-9, TIMP1, TIMP2, TIMP3, C-reactive protein, interleukin-1β, interleukin-1 receptor antagonist, interleukin-6, tumor necrosis factor α, soluble intercellular adhesion molecule-1, vascular cell adhesion molecule, monocyte chemotactic protein-1, caspase-3, human lipocalin-type prostaglandin D synthase, mast cell tryptase, eosinophil cationic protein, KL-6, procalcitonin, haptoglobin, s-CD40 ligand, S-FAS ligand, alpha 2 actin, basic calponin 1, CSRP2 elastin, LTBP4, smooth muscle myosin, smooth muscle myosin heavy chain, transgelin, aldosterone, angiotensin III, bradykinin, endothelin 1, endotehlin 2, endothelin 3, renin, APO B48, pancreatic elastase 1, pancreatic lipase, sPLA2, trypsinogen activation peptide, alpha enolase, LAMP3, phospholipase D, PLA2G5, protein D, SFTPC, defensin HBD1, defensin HBD2, CXCL-1, CXCL-2, CXCL-3, CCL2, CCL3, CCL4, CCL8, procalcitonin, protein C, serum amyloid A, s-glutathione, s-TNF P55, s-TNF P75, TAFI, TGF beta, MMP-11, brain fatty acid binding protein, CA11, CABP1, CACNA1A, CBLN1, CHN2, cleaved Tau, CRHR1, DRPLA, EGF, GPM6B, GPR7, GPR8, GRIN2C, GRM7, HAPIP, HIF 1 alpha, HIP2 KCNK4, KCNK9, KCNQ5, MAPK10, n-acetyl aspartate, NEUROD2, NRG2, PACE4, phosphoglycerate mutase, PKC gamma, prostaglandin E2, PTEN, PTPRZ1, RGS9, SCA7, secretagogin, SLC1A3, SORL1, SREB3, STAC, STX1A, STXBP1, BDNF, cystatin C, neurokinin A, substance P, interleukin-1, interleukin-11, interleukin-13, interleukin-18, interleukin-4, and interleukin-10.

The skilled artisan will also understand that the methods described herein are also applicable generally to identifying polypeptides, whether or not they are proteolytic fragments of another, larger, polypeptide, that share the ability to bind to an antibody of interest. Taking a known example, the polypeptide hormone cardiodilatin has a sequence that is identical to a portion of pro-ANP. Antibodies that bind to pro-ANP may, therefore, crossreact with cardiodilatin. If cardiodilatin was unknown in blood samples, this crossreactivity could be exploited to identify its presence by identifying those additional polypeptides that bind to the antibody.

Once unpredicted polypeptides that share the ability to bind to an antibody of interest are identified, their presence in serum may be characterized for use as disease markers as described hereinafter. In addition, antibodies may be selected to distinguish the various polypeptides. Returning to the caridodilatin/pro-ANP example above, if assays for pro-ANP had been shown to be related to a particular disease state, it may be that cardiodilatin was contributing to that relationship, or, in the alternative, confounding that relationship. Further characterization would now be possible, based on the knowledge that the antibody of interest was binding to more than the expected pro-ANP polypeptide.

Selection of Antibodies

The generation and selection of antibodies that recognize one or more natriuretic peptide fragments may be accomplished several ways. For example, one way is to purify the fragments of interest or to synthesize the fragments of interest using, e.g., solid phase peptide synthesis methods well known in the art. See, e.g., Guide to Protein Purfication, Murray P. Deutcher, ed., Meth. Enzymol. Vol 182 (1990); Solid Phase Peptide Synthesis, Greg B. Fields ed., Meth. Enzymol. Vol 289 (1997). One skilled in the art will recognize that regions that are common to a set of peptides may be used, rather than the entire fragment(s) of interest, to generate and/or identify antibodies that recognize the set of fragments containing that common region. Similarly, regions that are not in common between one or a set of fragment(s) may be used to generate and/or identify antibodies that distinguish between sets of fragments.

The selected polypeptides may then be injected, for example, into mice or rabbits, to generate polyclonal or monoclonal antibodies. One skilled in the art will recognize that many procedures are available for the production of antibodies, for example, as described in Antibodies, A Laboratory Manual, Ed Harlow and David Lane, Cold Spring Harbor Laboratory (1988), Cold Spring Harbor, N.Y. One skilled in the art will also appreciate that binding fragments or Fab fragments which mimic antibodies can also be prepared from genetic information by various procedures (Antibody Engineering: A Practical Approach (Borrebaeck, C., ed.), 1995, Oxford University Press, Oxford; J. Immunol. 149, 3914-3920 (1992)).

In addition, numerous publications have reported the use of phage display technology to produce and screen libraries of polypeptides for binding to a selected target. See, e.g, Cwirla et al., Proc. Natl. Acad. Sci. USA 87, 6378-82, 1990; Devlin et al., Science 249, 404-6, 1990, Scott and Smith, Science 249, 386-88, 1990; and Ladner et al., U.S. Pat. No. 5,571,698. A basic concept of phage display methods is the establishment of a physical association between DNA encoding a polypeptide to be screened and the polypeptide. This physical association is provided by the phage particle, which displays a polypeptide as part of a capsid enclosing the phage genome which encodes the polypeptide. The establishment of a physical association between polypeptides and their genetic material allows simultaneous mass screening of very large numbers of phage bearing different polypeptides. Phage displaying a polypeptide with affinity to a target bind to the target and these phage are enriched by affinity screening to the target. The identity of polypeptides displayed from these phage can be determined from their respective genomes. Using these methods a polypeptide identified as having a binding affinity for a desired target can then be synthesized in bulk by conventional means. See, e.g., U.S. Pat. No. 6,057,098, which is hereby incorporated in its entirety, including all tables, figures, and claims.

The antibodies that are generated by these methods mat then be selected by first screening for affinity and specificity with the purified natriuretic fragments of interest and, if required, comparing the results to the affinity and specificity of the antibodies with natriuretic fragments that are desired to be excluded from binding. The screening procedure can involve immobilization of the purified natriuretic fragments in separate wells of microtiter plates. The solution containing a potential antibody or groups of antibodies is then placed into the respective microtiter wells and incubated for about 30 min to 2 h. If an antibody to the fragment(s) of interest is present in the solution, it will bind to the immobilized natriuretic fragment(s). The microtiter wells are then washed and a labeled secondary antibody (for example, an anti-mouse antibody conjugated to alkaline phosphatase if the raised antibodies are mouse antibodies) is added to the wells and incubated for about 30 min and then washed. Substrate is added to the wells and a color reaction will appear where antibody to the immobilized natriuretic fragment(s) is present.

The antibodies so identified may then be further analyzed for affinity and specificity to the natriuretic fragment(s) of interest in the assay design selected. In the development of immunoassays for a target protein, the purified target protein acts as a standard with which to judge the sensitivity and specificity of the immunoassay using the antibodies that have been selected. Because the binding affinity of various antibodies for the various fragments may differ; certain antibody pairs (e.g., in sandwich assays) may interfere with one another sterically, etc., assay performance of an antibody may be a more important measure than absolute affinity and specificity of an antibody.

In another preferred embodiment, antibodies or binding fragments are directed to epitopes which are not changed by oxidation of methionine residues, or that can distinguish oxidized from reduced forms. The various oxidized and reduced forms of the polypeptides can be for generating and/or identifying antibodies as discussed above.

Those skilled in the art will recognize that many approaches can be taken in producing antibodies or binding fragments and screening and selecting for affinity and specificity for the various natriuretic peptide fragments, but these approaches do not change the scope of the invention.

Characterization of Natriuretic Peptide Fragments

Once antibodies to various regions of the natriuretic peptides have been obtained, these antibodies can be used to capture fragments from test samples for further characterization in order to identify the sequence of the various peptides present. Individual peptides may be obtained and sequenced using microsequencing methods known to the skilled artisan. See, e.g., A Practical Guide to Protein and Peptide Purification for Microsequencing, Paul T. Matsudaira, ed., Academic Press, San Diego, 1989. Peptide mass fingerprinting and amino acid analysis using mass spectrometry techniques are particularly well suited to identifying peptides so obtained. See, e.g., Westermeier and Naven, Proteomics in Practice: A Laboratory Manual of Proteome Analysis, Wiley-VCH Verlag-GmbH, Weinheim, 2002.

The terms “mass spectrometry” or “MS” as used herein refer to methods of filtering, detecting, and measuring ions based on their mass-to-charge ratio, or “m/z.” In general, one or more molecules of interest are ionized, and the ions are subsequently introduced into a mass spectrographic instrument where, due to a combination of magnetic and electric fields, the ions follow a path in space that is dependent upon mass (“m”) and charge (“z”). See, e.g., U.S. Pat. No. 6,204,500, entitled “Mass Spectrometry From Surfaces;” U.S. Pat. No. 6,107,623, entitled “Methods and Apparatus for Tandem Mass Spectrometry;” U.S. Pat. No. 6,268,144, entitled “DNA Diagnostics Based On Mass Spectrometry;” U.S. Pat. No. 6,124,137, entitled “Surface-Enhanced Photolabile Attachment And Release For Desorption And Detection Of Analytes;” Wright et al., “Proteinchip surface enhanced laser desorption/ionization (SELDI) mass spectrometry: a novel protein biochip technology for detection of prostate cancer biomarkers in complex protein mixtures,” Prostate Cancer and Prostatic Diseases 2: 264-76 (1999); and Merchant and Weinberger, “Recent advancements in surface-enhanced laser desorption/ionization-time of flight-mass spectrometry,” Electrophoresis 21: 1164-67 (2000), each of which is hereby incorporated by reference in its entirety, including all tables, figures, and claims.

For example, in a “quadrupole” or “quadrupole ion trap” instrument, ions in an oscillating radio frequency field experience a force proportional to the DC potential applied between electrodes, the amplitude of the RF signal, and m/z. The voltage and amplitude can be selected so that only ions having a particular m/z travel the length of the quadrupole, while all other ions are deflected. Thus, quadrupole instruments can act as both a “mass filter” and as a “mass detector” for the ions injected into the instrument.

Moreover, one can often enhance the resolution of the MS technique by employing “tandem mass spectrometry,” or “MS/MS.” In this technique, a precursor ion or group of ions generated from a molecule (or molecules) of interest may be filtered in an MS instrument, and these precursor ions subsequently fragmented to yield one or more fragment ions that are then analyzed in a second MS procedure. By careful selection of precursor ions, only ions produced by certain analytes of interest are passed to the fragmentation chamber, where collision with atoms of an inert gas occurs to produce the fragment ions. Because both the precursor and fragment ions are produced in a reproducible fashion under a given set of ionization/fragmentation conditions, the MS/MS technique can provide an extremely powerful analytical tool. For example, the combination of filtration/fragmentation can be used to eliminate interfering substances, and can be particularly useful in complex samples, such as biological samples.

Additionally, recent advances in technology, such as matrix-assisted laser desorption ionization coupled with time-of-flight analyzers (“MALDI-TOF”), or surface-enhanced laser desorption ionization coupled with time-of-flight analyzers (“SELDI-TOF”), permit the analysis of analytes at femtomole levels in very short ion pulses. Mass spectrometers that combine time-of-flight analyzers with tandem MS are also well known to the artisan. Additionally, multiple mass spectrometry steps can be combined in methods known as “MS/MS” and “MS/MS-TOF,” including MS/MS-MALDI-TOF and MS/MS-SELDI-TOF. Preferred apparatuses and methods for characterization and identification of proteins are disclosed in U.S. Patent Application Publication No. US 2002/0182649; U.S. Pat. No. 6,225,047; Issaq et al., Biochem. Biophys. Res. Commun. 292: 587-92 (2002); and Issaq et al., Anal. Chem. 75: 149A-155A (2003), each of which is hereby incorporated by reference in its entirety.

Ions can be produced using a variety of methods including, but not limited to, electron ionization, chemical ionization, fast atom bombardment, field desorption, and matrix-assisted laser desorption ionization (“MALDI”), surface enhanced laser desorption ionization (“SELDI”), photon ionization, electrospray ionization, and inductively coupled plasma.

Use of Natriuretic Peptide Degradation Products in Marker Panels

Methods and systems for the identification of a one or more markers for the diagnosis, and in particular for the differential diagnosis, of disease have been described previously. Suitable methods for identifying markers useful for the diagnosis of disease states are described in detail in U.S. patent application Ser. No. 10/331,127, entitled METHOD AND SYSTEM FOR DISEASE DETECTION USING MARKER COMBINATIONS (attorney docket no. 071949-6802), filed Dec. 27, 2002, which is hereby incorporated by reference in its entirety, including all tables, figures, and claims. One skilled in the art will also recognize that univariate analysis of markers can be performed and the data from the univariate analyses of multiple markers can be combined to form panels of markers to differentiate different disease conditions.

In developing a panel of markers useful in diagnosis, data for a number of potential markers may be obtained from a group of subjects by testing for the presence or level of certain markers. The group of subjects is divided into two sets, and preferably the first set and the second set each have an approximately equal number of subjects. The first set includes subjects who have been confirmed as having a disease or, more generally, being in a first condition state. For example, this first set of patients may be those that have recently had a disease incidence, or may be those having a specific type of disease. The confirmation of the condition state may be made through a more rigorous and/or expensive testing such as MRI or CT. Hereinafter, subjects in this first set will be referred to as “diseased”.

The second set of subjects are simply those who do not fall within the first set. Subjects in this second set may be “non-diseased;” that is, normal subjects. Alternatively, subjects in this second set may be selected to exhibit one symptom or a constellation of symptoms that mimic those symptoms exhibited by the “diseased” subjects. In still another alternative, this second set may represent those at a different time point from disease incidence.

The data obtained from subjects in these sets includes levels of a plurality of markers, including for purposes of the present invention, one or more fragments of natriuretic peptides either measured individually or as a group. Preferably, data for the same set of markers is available for each patient. This set of markers may include all candidate markers which may be suspected as being relevant to the detection of a particular disease or condition. Actual known relevance is not required. Embodiments of the methods and systems described herein may be used to determine which of the candidate markers are most relevant to the diagnosis of the disease or condition. The levels of each marker in the two sets of subjects may be distributed across a broad range, e.g., as a Gaussian distribution. However, no distribution fit is required.

A marker often is incapable of definitively identifying a patient as either diseased or non-diseased. For example, if a patient is measured as having a marker level that falls within the overlapping region, the results of the test will be useless in diagnosing the patient. An artificial cutoff may be used to distinguish between a positive and a negative test result for the detection of the disease or condition. Regardless of where the cutoff is selected, the effectiveness of the single marker as a diagnosis tool is unaffected. Changing the cutoff merely trades off between the number of false positives and the number of false negatives resulting from the use of the single marker. The effectiveness of a test having such an overlap is often expressed using a ROC (Receiver Operating Characteristic) curve. ROC curves are well known to those skilled in the art.

The horizontal axis of the ROC curve represents (1-specificity), which increases with the rate of false positives. The vertical axis of the curve represents sensitivity, which increases with the rate of true positives. Thus, for a particular cutoff selected, the value of (1-specificity) may be determined, and a corresponding sensitivity may be obtained. The area under the ROC curve is a measure of the probability that the measured marker level will allow correct identification of a disease or condition. Thus, the area under the ROC curve can be used to determine the effectiveness of the test.

As discussed above, the measurement of the level of a single marker may have limited usefulness. The measurement of additional markers provides additional information, but the difficulty lies in properly combining the levels of two potentially unrelated measurements. In the methods and systems according to embodiments of the present invention, data relating to levels of various markers for the sets of diseased and non-diseased patients may be used to develop a panel of markers to provide a useful panel response. The data may be provided in a database such as Microsoft Access, Oracle, other SQL databases or simply in a data file. The database or data file may contain, for example, a patient identifier such as a name or number, the levels of the various markers present, and whether the patient is diseased or non-diseased.

Next, an artificial cutoff region may be initially selected for each marker. The location of the cutoff region may initially be selected at any point, but the selection may affect the optimization process described below. In this regard, selection near a suspected optimal location may facilitate faster convergence of the optimizer. In a preferred method, the cutoff region is initially centered about the center of the overlap region of the two sets of patients. In one embodiment, the cutoff region may simply be a cutoff point. In other embodiments, the cutoff region may have a length of greater than zero. In this regard, the cutoff region may be defined by a center value and a magnitude of length. In practice, the initial selection of the limits of the cutoff region may be determined according to a pre-selected percentile of each set of subjects. For example, a point above which a pre-selected percentile of diseased patients are measured may be used as the right (upper) end of the cutoff range.

Each marker value for each patient may then be mapped to an indicator. The indicator is assigned one value below the cutoff region and another value above the cutoff region. For example, if a marker generally has a lower value for non-diseased patients and a higher value for diseased patients, a zero indicator will be assigned to a low value for a particular marker, indicating a potentially low likelihood of a positive diagnosis. In other embodiments, the indicator may be calculated based on a polynomial. The coefficients of the polynomial may be determined based on the distributions of the marker values among the diseased and non-diseased subjects.

The relative importance of the various markers may be indicated by a weighting factor. The weighting factor may initially be assigned as a coefficient for each marker. As with the cutoff region, the initial selection of the weighting factor may be selected at any acceptable value, but the selection may affect the optimization process. In this regard, selection near a suspected optimal location may facilitate faster convergence of the optimizer. In a preferred method, acceptable weighting coefficients may range between zero and one, and an initial weighting coefficient for each marker may be assigned as 0.5. In a preferred embodiment, the initial weighting coefficient for each marker may be associated with the effectiveness of that marker by itself. For example, a ROC curve may be generated for the single marker, and the area under the ROC curve may be used as the initial weighting coefficient for that marker.

Next, a panel response may be calculated for each subject in each of the two sets. The panel response is a function of the indicators to which each marker level is mapped and the weighting coefficients for each marker. In a preferred embodiment, the panel response (R) for a each subject (j) is expressed as:

Rⱼ=ΣwᵢIᵢⱼ,

where i is the marker index, j is the subject index, wᵢ is the weighting coefficient for marker i, I is the indicator value to which the marker level for marker i is mapped for subject j, and Σ is the summation over all candidate markers i.

One advantage of using an indicator value rather than the marker value is that an extraordinarily high or low marker levels do not change the probability of a diagnosis of diseased or non-diseased for that particular marker. Typically, a marker value above a certain level generally indicates a certain condition state. Marker values above that level indicate the condition state with the same certainty. Thus, an extraordinarily high marker value may not indicate an extraordinarily high probability of that condition state. The use of an indicator which is constant on one side of the cutoff region eliminates this concern.

The panel response may also be a general function of several parameters including the marker levels and other factors including, for example, race and gender of the patient. Other factors contributing to the panel response may include the slope of the value of a particular marker over time. For example, a patient may be measured when first arriving at the hospital for a particular marker. The same marker may be measured again an hour later, and the level of change may be reflected in the panel response. Further, additional markers may be derived from other markers and may contribute to the value of the panel response. For example, the ratio of values of two markers may be a factor in calculating the panel response.

Having obtained panel responses for each subject in each set of subjects, the distribution of the panel responses for each set may now be analyzed. An objective function may be defined to facilitate the selection of an effective panel. The objective function should generally be indicative of the effectiveness of the panel, as may be expressed by, for example, overlap of the panel responses of the diseased set of subjects and the panel responses of the non-diseased set of subjects. In this manner, the objective function may be optimized to maximize the effectiveness of the panel by, for example, minimizing the overlap.

In a preferred embodiment, the ROC curve representing the panel responses of the two sets of subjects may be used to define the objective function. For example, the objective function may reflect the area under the ROC curve. By maximizing the area under the curve, one may maximize the effectiveness of the panel of markers. In other embodiments, other features of the ROC curve may be used to define the objective function. For example, the point at which the slope of the ROC curve is equal to one may be a useful feature. In other embodiments, the point at which the product of sensitivity and specificity is a maximum, sometimes referred to as the “knee,” may be used. In an embodiment, the sensitivity at the knee may be maximized. In further embodiments, the sensitivity at a predetermined specificity level may be used to define the objective function. Other embodiments may use the specificity at a predetermined sensitivity level may be used. In still other embodiments, combinations of two or more of these ROC-curve features may be used.

It is possible that one of the markers in the panel is specific to the disease or condition being diagnosed. When such markers are present at above or below a certain threshold, the panel response may be set to return a “positive” test result. When the threshold is not satisfied, however, the levels of the marker may nevertheless be used as possible contributors to the objective function.

An optimization algorithm may be used to maximize or minimize the objective function. Optimization algorithms are well-known to those skilled in the art and include several commonly available minimizing or maximizing functions including the Simplex method and other constrained optimization techniques. It is understood by those skilled in the art that some minimization functions are better than others at searching for global minimums, rather than local minimums. In the optimization process, the location and size of the cutoff region for each marker may be allowed to vary to provide at least two degrees of freedom per marker. Such variable parameters are referred to herein as independent variables. In a preferred embodiment, the weighting coefficient for each marker is also allowed to vary across iterations of the optimization algorithm. In various embodiments, any permutation of these parameters may be used as independent variables.

In addition to the above-described parameters, the sense of each marker may also be used as an independent variable. For example, in many cases, it may not be known whether a higher level for a certain marker is generally indicative of a diseased state or a non-diseased state. In such a case, it may be useful to allow the optimization process to search on both sides. In practice, this may be implemented in several ways. For example, in one embodiment, the sense may be a truly separate independent variable which may be flipped between positive and negative by the optimization process. Alternatively, the sense may be implemented by allowing the weighting coefficient to be negative.

The optimization algorithm may be provided with certain constraints as well. For example, the resulting ROC curve may be constrained to provide an area-under-curve of greater than a particular value. ROC curves having an area under the curve of 0.5 indicate complete randomness, while an area under the curve of 1.0 reflects perfect separation of the two sets. Thus, a minimum acceptable value, such as 0.75, may be used as a constraint, particularly if the objective function does not incorporate the area under the curve. Other constraints may include limitations on the weighting coefficients of particular markers. Additional constraints may limit the sum of all the weighting coefficients to a particular value, such as 1.0.

The iterations of the optimization algorithm generally vary the independent parameters to satisfy the constraints while minimizing or maximizing the objective function. The number of iterations may be limited in the optimization process. Further, the optimization process may be terminated when the difference in the objective function between two consecutive iterations is below a predetermined threshold, thereby indicating that the optimization algorithm has reached a region of a local minimum or a maximum.

Thus, the optimization process may provide a panel of markers including weighting coefficients for each marker and cutoff regions for the mapping of marker values to indicators. In order to develop lower-cost panels which require the measurement of fewer marker levels, certain markers may be eliminated from the panel. In this regard, the effective contribution of each marker in the panel may be determined to identify the relative importance of the markers. In one embodiment, the weighting coefficients resulting from the optimization process may be used to determine the relative importance of each marker. The markers with the lowest coefficients may be eliminated.

In certain cases, the lower weighting coefficients may not be indicative of a low importance. Similarly, a higher weighting coefficient may not be indicative of a high importance. For example, the optimization process may result in a high coefficient if the associated marker is irrelevant to the diagnosis. In this instance, there may not be any advantage that will drive the coefficient lower. Varying this coefficient may not affect the value of the objective function.

Use of BNP and its Fragments for Determining a Treatment Regimen

A useful diagnostic or prognostic indicator, such as the natriuretic peptide fragments described herein, can help clinicians select between alternative therapeutic regimens. For example, patients with elevation in cardiac troponin T or I following an acute coronary syndrome appear to derive specific benefit from an early aggressive strategy that includes potent antiplatelet and antithrombotic therapy, and early revascularization. Hamm et al., N. Engl. J. Med. 340: 1623-9 (1999); Morrow et al., J. Am. Coll. Cardiol. 36: 1812-7 (2000); Cannon et al., Am. J. Cardiol. 82: 731-6 (1998). Additionally, patients with elevation in C-reactive protein following myocardial infarction appear to derive particular benefit from HMG-CoA Reductase Inhibitor therapy. Ridker et al., Circulation 98: 839-44 (1998). Among patients with congestive heart failure, pilot studies suggest that ACE inhibitors may reduce BNP levels in a dose dependent manner. Van Veldhuisen et al., J. Am. Coll. Cardiol. 32: 1811-8 (1998).

Similarly, “tailoring” diuretic and vasodilator therapy based on the level of the various natriuretic peptide fragments may improve outcomes. See, e.g., Troughton et al., Lancet 355: 1126-30 (2000). Finally, in a single pilot study of 16 patients found that randomization to an ACE inhibitor rather than placebo following Q-wave MI was associated with reduced BNP levels over the subsequent. 6-month period. Motwani et al., Lancet 341: 1109-13 (1993). Because BNP is a counter-regulatory hormone with beneficial cardiac and renal effects, it is likely that a change in BNP concentration reflects improved ventricular function and reduced ventricular wall stress. A recent article demonstrates the correlation of NT pro-BNP and BNP assays (Fischer et al., Clin. Chem. 47: 591-594 (2001). It is a further objective of this invention that the concentration of natriuretic peptide fragments, either individually or considered in groups, can be used to guide diuretic and vasodilator therapy to improve patient outcome. Additionally, the measurement of natriuretic peptide fragments, either individually or considered in groups, for use as a prognostic indicator for patients suffering from acute coronary syndromes, is within the scope of the present invention.

Recent studies in patients hospitalized with congestive heart failure suggest that serial BNP measurements may provide incremental prognositic information as compared to a single measurement; that is, assays can demonstrate an improving prognosis when BNP falls after therapy than when it remains persistently elevated. Cheng et al., J. Am. Coll. Cardiol. 37: 386-91 (2001). Thus, serial measurements of natriuretic peptide fragments may increase the prognostic and/or diagnostic value of a marker in patients, and is thus within the scope of the present invention.

Assay Measurement Srategies

Numerous methods and devices are well known to the skilled artisan for the detection and analysis of polypeptides or proteins in test samples. In preferred embodiments, immunoassay devices and methods are often used. See, e.g., U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792, each of which is hereby incorporated by reference in its entirety, including all tables, figures and claims. These devices and methods can utilize labeled molecules in various sandwich, competitive, or non-competitive assay formats, to generate a signal that is related to the presence or amount of an analyte of interest. Additionally, certain methods and devices, such as biosensors and optical immunoassays, may be employed to determine the presence or amount of analytes without the need for a labeled molecule. See, e.g., U.S. Pat. Nos. 5,631,171; and 5,955,377, each of which is hereby incorporated by reference in its entirety, including all tables, figures and claims. One skilled in the art also recognizes that robotic instrumentation including but not limited to Beckman Access, Abbott AxSym, Roche ElecSys, Dade Behring Stratus systems are among the immunoassay analyzers that are capable of performing the immunoassays taught herein. Specific immunological binding of the antibody to the marker can be detected directly or indirectly. Direct labels include fluorescent or luminescent tags, metals, dyes, radionuclides, and the like, attached to the antibody. Indirect labels include various enzymes well known in the art, such as alkaline phosphatase, horseradish peroxidase and the like.

The use of immobilized antibodies specific for the one or more polypeptides is also contemplated by the present invention. The antibodies could be immobilized onto a variety of solid supports, such as magnetic or chromatographic matrix particles, the surface of an assay place (such as microtiter wells), pieces of a solid substrate material or membrane (such as plastic, nylon, paper), and the like. An assay strip could be prepared by coating the antibody or a plurality of antibodies in an array on solid support. This strip could then be dipped into the test sample and then processed quickly through washes and detection steps to generate a measurable signal, such as a colored spot.

The analysis of a plurality of polypeptides may be carried out separately or simultaneously with one test sample. For separate or sequential assay, suitable apparatuses include clinical laboratory analyzers such as the ElecSys (Roche), the AxSym (Abbott), the Access (Beckman), the ADVIA® CENTAUR® (Bayer) immunoassay systems, the NICHOLS ADVANTAGE® (Nichols Institute) immunoassay system, etc. Preferred apparatuses or protein chips perform simultaneous assays of a plurality of polypeptides on a single surface. Particularly useful physical formats comprise surfaces having a plurality of discrete, adressable locations for the detection of a plurality of different analytes. Such formats include protein microarrays, or “protein chips” (see, e.g., Ng and Ilag, J. Cell Mol. Med. 6: 329-340 (2002)) and certain capillary devices (see, e.g., U.S. Pat. No. 6,019,944). In these embodiments, each discrete surface location may comprise antibodies to immobilize one or more analyte(s) (e.g., one or more polypeptides of the invention) for detection at each location. Surfaces may alternatively comprise one or more discrete particles (e.g., microparticles or nanoparticles) immobilized at discrete locations of a surface, where the microparticles comprise antibodies to immobilize one analyte (e.g., one or more polypeptides of the invention) for detection.

In addition, one skilled in the art would recognize the value of testing multiple samples (for example, at successive time points) from the same individual. Such testing of serial samples will allow the identification of changes in polypeptide levels over time. Increases or decreases in polypeptide levels, as well as the absence of change in such levels, would provide useful information about the disease status that includes, but is not limited to identifying the approximate time from onset of the event, the presence and amount of salvagable tissue, the appropriateness of drug therapies, the effectiveness of various therapies as indicated by reperfusion or resolution of symptoms, differentiation of the various types of disease having similar symptoms, identification of the severity of the event, identification of the disease severity, and identification of the patient's outcome, including risk of future events.

A panel consisting of the polypeptides referenced above, and optionally including other protein markers useful in diagnosis, prognosis, or differentiation of disease, may be constructed to provide relevant information related to differential diagnosis. Such a panel may be constructed to detect 1, 2, 3, 4, 5, 6, 7, 8, 9, 10, 15, 20, or more or individual analytes, including one or more polypeptides of the present invention. The analysis of a single analyte or subsets of analytes could be carried out by one skilled in the art to optimize clinical sensitivity or specificity in various clinical settings. These include, but are not limited to ambulatory, urgent care, critical care, intensive care, monitoring unit, inpatient, outpatient, physician office, medical clinic, and health screening settings. Furthermore, one skilled in the art can use a single analyte or a subset of analytes in combination with an adjustment of the diagnostic threshold in each of the aforementioned settings to optimize clinical sensitivity and specificity. The clinical sensitivity of an assay is defined as the percentage of those with the disease that the assay correctly predicts, and the specificity of an assay is defined as the percentage of those without the disease that the assay correctly predicts (Tietz Textbook of Clinical Chemistry, 2^{nd} edition, Carl Burtis and Edward Ashwood eds., W. B. Saunders and Company, p. 496).

The analysis of analytes could be carried out in a variety of physical formats as well. For example, the use of microtiter plates or automation could be used to facilitate the processing of large numbers of test samples. Alternatively, single sample formats could be developed to facilitate immediate treatment and diagnosis in a timely fashion, for example, in ambulatory transport or emergency room settings.

As discussed above, samples may continue to degrade the natriuretic peptides or fragments thereof, even once the sample is obtained. Thus, it may be advantageous to add one or more protease inhibitors to samples prior to assay. Numerous protease inhibitors are known to those of skill in the art, and exemplary inhibitors may be found in, e.g., The Complete Guide for Protease Inhibition, Roche Molecular Biochemicals, updated Jun. 3, 1999 at http://www.roche-applied-science.com/fst/products.htm?/prod_inf/manuals/protease/prot_toc.htm, which is hereby incorporated in its entirety. Because various metalloproteases and calcium-dependent proteases are known to exist in blood-derived samples, chelators such as EGTA and/or EDTA, also act as protease inhibitors.

### EXAMPLES

The following examples serve to illustrate the present invention. These examples are in no way intended to limit the scope of the invention.

### Example 1

### Blood Sampling

Blood is preferably collected by venous puncture using a 20 gauge multi-sample needle and evacuated tubes, although fingertip puncture, plantar surface puncture, earlobe puncture, etc., may suffice for small volumes. For whole blood collection, blood specimens are collected by trained study personnel in EDTA-containing blood collection tubes. For serum collection, blood specimens are collected by trained study personnel in thrombin-containing blood collection tubes. Blood is allowed to clot for 5-10 minutes, and serum is separated from insoluble material by centrifugation. For plasma collection, blood specimens are collected by trained study personnel in citrate-containing blood collection tubes and centrifuged for 12 minutes. Samples may be kept at 4° C. until use, or frozen at −20° C. or colder for longer term storage. Whole blood is preferably not frozen.

### Example 2

### Biochemical Analyses

BNP is measured using standard immunoassay techniques. These techniques involve the use of antibodies to specifically bind the protein targets. An antibody directed against BNP is biotinylated using N-hydroxysuccinimide biotin (NH S-biotin) at a ratio of about 5 NHS-biotin moieties per antibody. The biotinylated antibody is then added to wells of a standard avidin 384 well microtiter plate, and biotinylated antibody not bound to the plate is removed. This formed an anti-BNP solid phase in the microtiter plate. Another anti-BNP antibody is conjugated to alkaline phosphatase using standard techniques, using SMCC and SPDP (Pierce, Rockford, Ill.). The immunoassays are performed on a TECAN Genesis RSP 200/8 Workstation. Test samples (10 μL) are pipeted into the microtiter plate wells, and incubated for 60 min. The sample is then removed and the wells washed with a wash buffer, consisting of 20 mM borate (pH 7.42) containing 150 mM NaCl, 0.1% sodium azide, and 0.02% Tween-20. The alkaline phosphatase-antibody conjugate is then added to the wells and incubated for an additional 60 min, after which time, the antibody conjugate is removed and the wells washed with a wash buffer. A substrate, (AttoPhos®, Promega, Madison, Wis.) is added to the wells, and the rate of formation of the fluorescent product is related to the concentration of the BNP in the test samples.

### Example 3

### Identification of BNP Peptides in Spiked Test Samples

Purified BNP (either BNP₁₋₁₀₈ or BNP₇₇₋₁₀₈) is added to human blood, serum and plasma test samples, and allowed to incubate for from 5 minutes to 24 hours minutes at 22° C. Following this incubation, the samples are subjected to the following analysis to identify BNP-derived peptides present in the samples.

Test samples were analyzed using a chip-based platform (Ciphergen Biosystems ProteinChip®) coated with anti-BNP antibodies (mouse monoclonal or recombinant human antibodies). For preparing the surface, Protein A or Protein G from Staphylococcus species or Protein D from Haemophilus species is immobilized to an epoxide on a PS2 ProteinChip® surface by incubation for 2 hours in a humid chamber at room temperature. Residual epoxide sites are blocked with 0.5M ethanolamine in phosphate buffered saline (PBS), pH 8.0 for 15 minutes, then the ProteinChip® is washed 1× with 0.5% Triton X-100 in PBS and 3× in PBS for 15 minutes each. The ProteinChip® is air dried. About 2 μL of each desired antibody is applied to individual array locations at 2-3 mg/mL. The chip is incubated in a humid environment for 1-10 hours. The ProteinChip® is washed 1× with 0.5% Triton X-100 in PBS and 3× in PBS for 15 minutes each, air dried, and is ready for use.

The array locations are exposed to sample for from 10 minutes to 24 hours in a humid environment at room temperature. Unbound material is removed by washing in one or more suitable buffers selected to provide a desired level of stringency (that is, removal of material bound at lower affinity, such as nonspecific background binding). Suitable buffers include PBS; PBS containing 0.05% v/v Tween 20; PBS containing 0.1-3M urea; 20 mM borate (pH 7.42) containing 150 mM NaCl, 0.1% sodium azide, and 0.02% Tween-20; and 0.1M urea, 50 mM CHAPS, 150 mM KCl, pH 7-8. This list is not meant to be limiting, and additional buffers can readily be selected for use by those of skill in the art.

SELDI-TOF-MS is used to determine the identity of polypeptides bound to the anti-BNP antibodies by mass analysis. See, e.g., U.S. Pat. Nos. 5,719,060; 5,894,063; 6,020,208; 6,027,942; and 6,124,137, each of which is hereby incorporated in its entirety, including all tables, figures, and claims. Following drying of the surface, a matrix solution is applied (e.g., sinapinic acid). Each array location is subsequently interrogated with a laser desorption/ionization source, and the ions generated analyzed by SELDI-TOF. Peptide ID is obtained by matching an observed m/z to a predicted molecular weight. Additional resolution can be obtained using the MS/MS methods disclosed in U.S. Patent Application Publication No. US 2002/0182649, which is incorporated by reference herein.

The following BNP fragments were identified in spiked plasma samples: BNP₇₇₋₁₀₆; BNP₇₉₋₁₀₆; BNP₇₉₋₁₀₈; BNP₇₇₋₁₀₈; BNP₆₉₋₁₀₀; BNP₇₆₋₁₀₇; BNP₃₉₋₈₆; BNP₅₃₋₈₅; BNP₆₆₋₉₈; BNP₃₀₋₁₀₃; BNP₁₁₋₁₀₇; and BNP₉₋₁₀₆. In addition, methionine oxidation could be observed as a 15-16 Dalton increase from the predicted molecular weight of a given fragment. Significant oxidation of one or two methionines could be observed in those fragments containing methionine residues. Moreover, a “total BNP” measurement obtained by summation of the area under the peaks of observed fragments indicated that not all of the BNP added was being detected by the antibodies used. This leads to the conclusion that BNP fragments are present in these samples.

### Example 4

### Identification of BNP Peptides in Patient Test Samples

Plasma, serum, or blood samples obtained from seven human patients presenting for clinical evaluation of chest pain are subjected to the same analysis described in Example 3. Initial patient screening is performed by trained medical personnel, and a clinical diagnosis is obtained by conventional medical means. Plasma samples are obtained from each patient at clinical presentation, and an “apparent BNP” concentration measured by immunoassay, using purified BNP as a standard.

A summary of results for 10 patients is provided in the following table:

| Patient | Clinical Diagnosis | Apparent BNP (pg/mL) |
|---|---|---|
| 22085 | Unstable angina | 39.6 |
| 22995 | Non-cardiac chest pain | 161 |
| 21231 | Unstable angina | 353.5 |
| 16221 | Acute myocardial infarction | 654.8 |
| 9240 | Congestive heart failure, | 905.5 |
| | diastolic dysfunction | |
| 9842 | Echo ejection fraction 44%, | 1588.7 |
| | enlarged left atrium/ventricle | |
| 21221 | Hospitalization for hyperkalemia | 3561.9 |
| 8329 | Class IV Congestive heart failure | 1207.3 |
| 5478 | Isehemic stroke | 2410.6 |
| 10323 | Subarrachnoid hemorrhage | 591.9 |

The following BNP fragments were identified in plasma samples from the various samples: BNP₃₋₁₀₈; BNP₇₇₋₁₀₈; BNP₇₉₋₁₀₈; BNP₈₀₋₁₀₈; BNP₈₁₋₁₀₈; and BNP₈₃₋₁₀₈. Additional peaks, which have not yet been related to a BNP sequence, are seen at the following molecular weights: about 2576; about 2676; about 2792; about 3154; about 3370. Additional unidentified polypeptides were also captured by the antibodies.

In addition, a fragment corresponding to the molecular weight of a tetrameric BNP₇₇₋₁₀₈ was also observed in certain samples (m/z about 12,900). While not wishing to be bound to a particular mechanism, thiol-disulfide interchanges have been reported in proteins including acetyleholinesterase. The disulfide exchange reaction originates from nucleophilic attack on a sulfur atom of the disulfide by the free thiol. As BNP₇₇₋₁₀₈ contains cysteine residues that ordinarily participate in intramolecular disulfide bond formation, high concentrations of mature BNP formation could result in formation of multimeric forms by interaction of reduced and oxidized BNP forms.

In addition, variations in the BNP fragments were observed that were diagnosis-dependent. For example, patient 21231 exhibited a high level of observable BNP3-108 and an intermediate “apparent BNP” concentration, while patient 9240 exhibited little BNP3-108 despite a much higher “apparent BNP” concentration. Thus, BNP3-108, either alone or together with a BNP concentration reflective of a number of additional fragments being bound by the antibody may distinguish unstable angina or myocardial infarction from congestive heart failure.

While the invention has been described and exemplified in sufficient detail for those skilled in this art to make and use it, various alternatives, modifications, and improvements should be apparent without departing from the spirit and scope of the invention.

One skilled in the art readily appreciates that the present invention is well adapted to carry out the objects and obtain the ends and advantages mentioned, as well as those inherent therein. The examples provided herein are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

Other embodiments are set forth within the following claims.

We claim:

1. A method of standardizing an immunoassay to determine the presence or amount of one or more analytes in a test sample, comprising:
determining a control immunoassay signal that is related to the presence or amount in a standard sample of one or more substantially purified human natriuretic peptide fragments other than mature ANP, mature BNP, mature CNP, cardiodilatin, pre-pro-ANP, pre-pro-BNP, pre-pro-CNP, pro-ANP, pro-BNP, pro-CNP, or a fragment corresponding to that obtained by removal of mature ANP, BNP, or CNP from pro-ANP, pro-BNP, or pro-CNP; and
using said control immunoassay signal to correlate a test immunoassay signal related to the presence or amount of one of said analyte(s) in said test sample to an immunoassay result.

2. A method according to claim 1, wherein said standard sample comprises one or more fragments of BNP₁₋₁₀₈ other than BNP₁₋₁₀₈, BNP₁₋₇₆ or BNP₇₇₋₁₀₈.

3. A method according to claim 1, wherein said standard sample comprises one or more fragments of BNP₇₇₋₁₀₈ other than BNP₇₇₋₁₀₈.

4. A method according to claim 1, wherein said standard sample comprises one or more fragments of BNP₁₋₁₀₈ selected from the group consisting of BNP₇₇₋₁₀₆, BNP₇₉₋₁₀₆, BNP₇₆₋₁₀₇, BNP₆₉₋₁₀₈, BNP₇₉₋₁₀₈, BNP₈₀₋₁₀₈, BNP₈₁₋₁₀₈, BNP₈₃₋₁₀₈, BNP₃₉₋₈₆, BNP₅₃₋₈₅, BNP₆₆₋₉₈, BNP₃₀₋₁₀₃, BNP₁₁₋₁₀₇, BNP₉₋₁₀₆, and BNP₃₋₁₀₈.

5. A method according to claim 1, wherein said test sample is a human sample.

6. A method according to claim 5, wherein said test sample is blood, plasma, or serum.

7. A method of selecting an antibody, comprising:
determining a plurality of fragments of pro-ANP, pro-BNP, or pro-CNP to be bound by said antibody that are naturally present in human blood, serum or plasma, wherein one or more of said fragments are not mature ANP, mature BNP, mature CNP, cardiodilatin, pre-pro-ANP, pre-pro-BNP, pre-pro-CNP, pro-ANP, pro-BNP, pro-CNP, or a fragment corresponding to that obtained by removal of mature ANP, BNP, or CNP from pro-ANP, pro-BNP, or pro-CNP; and identifying one or more antibodies that bind said plurality of fragments to provide said selected antibody.

8. A method according to claim 7, wherein said selected antibody is obtained by pooling a plurality of individual antibodies.

9. A method according to claim 7, wherein said selected antibody is obtained by selecting an antibody that binds to a region common to each member of said plurality of fragments.

10. A method according to claim 7, wherein said selected antibody is a monoclonal antibody.

11. A method according to claim 7, wherein said selected antibody is identified by phage display.

12. A method according to claim 7, wherein said selected antibody is an Omniclonal antibody.

13. A method according to claim 7, wherein said method further comprises binding said selected antibody to a solid phase.

14. A method according to claim 7, wherein said method further comprises conjugating said selected antibody to a detectable label.

15. A method according to claim 7, wherein said plurality of fragments are a plurality of fragments of pre-pro-BNP,

16. A method according to claim 15, wherein said plurality of fragments comprise a plurality of fragments of BNP₁₋₁₀₈.

17. A method according to claim 15, wherein said plurality of fragments comprise a plurality of fragments of BNP₇₇₋₁₀₈.

18. A method according to claim 15, wherein said plurality of fragments comprise one or more fragments of BNP₁₋₁₀₈ selected from the group consisting of BNP₇₇₋₁₀₆, BNP₇₉₋₁₀₆, BNP₇₆₋₁₀₇, BNP₆₉₋₁₀₈, BNP₇₉₋₁₀₈, BNP₈₀₋₁₀₈, BNP₈₁₋₁₀₈, BNP₈₃₋₁₀₈, BNP₃₉₋₈₆, BNP₅₃₋₈₅, BNP₆₆₋₉₈, BNP₃₀₋₁₀₃, BNP₁₁₋₁₀₇, BNP₉₋₁₀₆, and BNP₃₋₁₀₈.

19. A method of selecting an antibody, comprising:
selecting one or more antibodies that distinguish between a first group comprising one or more fragments of pre-pro-ANP, pre-pro-BNP, or pre-pro-CNP, and a second group comprising one or more different fragments of pre-pro-ANP, pre-pro-BNP, or pre-pro-CNP, wherein one or both of said first group and said second group comprise one or more fragments naturally present in human blood, serum or plasma that are not mature ANP, mature BNP, mature CNP, cardiodilatin, pre-pro-ANP, pre-pro-BNP, pre-pro-CNP, pro-ANP, pro-BNP, pro-CNP, or a fragment corresponding to that obtained by removal of mature ANP, BNP, or CNP from pro-ANP, pro-BNP, or pro-CNP.

20. A method according to claim 19, wherein said selected antibody is obtained by pooling a plurality of individual antibodies.

21. A method according to claim 19, wherein said selected antibody is obtained by selecting an antibody that binds to a region that is common to members of said first group, and that is absent from the members of said second group.

22. A method according to claim 19, wherein said selected antibody is a monoclonal antibody.

23. A method according to claim 19, wherein said selected antibody is identified by phage display.

24. A method according to claim 19, wherein said method further comprises binding said selected antibody to a solid phase.

25. A method according to claim 19, wherein said method further comprises conjugating said selected antibody to a detectable label.

26. A method according to claim 19, wherein said first group and said second group each comprise fragments of BNP₁₋₁₀₈.

27. A method according to claim 19, wherein said first group and said second group each comprise fragments of BNP₇₇₋₁₀₈.

28. A method according to claim 19, wherein said first group comprises one or more fragments of BNP₁₋₁₀₈ selected from the group consisting of BNP₇₇₋₁₀₆, BNP₇₉₋₁₀₆, BNP₇₆₋₁₀₇, BNP₆₉₋₁₀₈, BNP₇₉₋₁₀₈, BNP₈₀₋₁₀₈, BNP₈₁₋₁₀₈, BNP₈₃₋₁₀₈, BNP₃₉₋₈₆, BNP₅₃₋₈₅, BNP₆₆₋₉₈, BNP₃₀₋₁₀₃, BNP₁₁₋₁₀₇, BNP₉₋₁₀₆, and BNP₃₋₁₀₈.

29. A method according to claim 28, wherein said second group comprises one or more fragments of BNP₁₋₁₀₈ selected from the group consisting of BNP₇₇₋₁₀₆, BNP₇₉₋₁₀₆, BNP₇₆₋₁₀₇, BNP₆₉₋₁₀₈, BNP₇₉₋₁₀₈, BNP₈₀₋₁₀₈, BNP₈₁₋₁₀₈, BNP₈₃₋₁₀₈, BNP₃₉₋₈₆, BNP₅₃₋₈₅, BNP₆₆₋₉₈, BNP₃₀₋₁₀₃, BNP₁₁₋₁₀₇, BNP₉₋₁₀₆, and BNP₃₋₁₀₈.

30. A method of determining the presence or amount of a plurality of natriuretic peptide fragments of interest in a human blood, serum, or plasma test sample, comprising:
performing an immunoassay using an antibody that binds to a plurality of fragments of pro-ANP, pro-BNP, or pro-CNP naturally present in human blood, serum or plasma, one or more of which is not mature ANP, mature BNP, mature CNP, cardiodilatin, pre-pro-ANP, pre-pro-BNP, pre-pro-CNP, pro-ANP, pro-BNP, pro-CNP, or a fragment corresponding to that obtained by removal of mature ANP, BNP, or CNP from pro-ANP, pro-BNP, or pro-CNP, wherein a signal from said immunoassay depends upon binding to said antibody; and
relating a signal from said immunoassay to the presence or amount of said natriuretic peptide fragments of interest in said test sample.

31. A method according to claim 30, wherein said antibody is obtained by pooling a plurality of individual antibodies.

32. A method according to claim 30, wherein said antibody is obtained by selecting an antibody that binds to a region that is common to said plurality of fragments.

33. A method according to claim 30, wherein said antibody is a monoclonal antibody.

34. A method according to claim 30, wherein said antibody is obtained by phage display.

35. A method according to claim 30, wherein said antibody is bound to a solid phase.

36. A method according to claim 30, wherein said antibody is conjugated to a detectable label.

37. A method according to claim 30, wherein said plurality of fragments comprise fragments of BNP₇₇₋₁₀₈.

38. A method according to claim 37, wherein said plurality of fragments comprise fragments of BNP₇₇₋₁₀₈.

39. A method according to claim 37, wherein said plurality of fragments comprises one or more fragments of BNP₁₋₁₀₈ selected from the group consisting of BNP₇₇₋₁₀₆, BNP₇₉₋₁₀₆, BNP₇₆₋₁₀₇, BNP₆₉₋₁₀₈, BNP₇₉₋₁₀₈, BNP₈₀₋₁₀₈, BNP₈₁₋₁₀₈, BNP₈₃₋₁₀₈, BNP₃₉₋₈₆, BNP₅₃₋₈₅, BNP₆₆₋₉₈, BNP₃₀₋₁₀₃, BNP₁₁₋₁₀₇, BNP₉₋₁₀₆, and BNP₃₋₁₀₈.

40. A method according to claim 38, wherein said plurality of fragments comprises one or more fragments of BNP₁₋₁₀₈ selected from the group consisting of BNP₇₇₋₁₀₆, BNP₇₉₋₁₀₆, BNP₇₆₋₁₀₇, BNP₇₉₋₁₀₈, BNP₈₀₋₁₀₈, BNP₈₋₁₀₈, and BNP₈₃₋₁₀₈.

41. A method according to claim 30, further comprising correlating the presence or amount of said natriuretic peptide fragments of interest to the presence or absence of a disease in the human from which the test sample is obtained.

42. A method according to claim 41, wherein said disease is selected from the group consisting of stroke, congestive heart failure (CHF), cardiac ischemia, systemic hypertension, acute coronary syndrome, and acute myocardial infarction.

43. A method according to claim 30, further comprising correlating the presence or amount of said natriuretic peptide of interest to the probability of a future adverse event in the human from which the test sample is obtained.

44. A method according to claim 43, wherein said future adverse event is selected from the group consisting of vascular injury caused by cerebral vasospasm, onset of delayed neurologic deficit in a patient after stroke, death, myocardial infarction, and congestive heart failure.

45. A substantially purified polypeptide selected from the group consisting of BNP₇₇₋₁₀₆, BNP₇₉₋₁₀₆, BNP₇₆₋₁₀₇, BNP₆₉₋₁₀₈, BNP₈₁₋₁₀₈, BNP₈₃₋₁₀₈, BNP₃₉₋₈₆, BNP₅₃₋₈₅, BNP₆₆₋₉₈, BNP₃₀₋₁₀₃, BNP₁₁₋₁₀₇, BNP₉₋₁₀₆, and BNP₃₋₁₀₈.

46. A method of identifying one or more unpredicted polypeptides structurally related to a known polypeptide in a biological sample, the method comprising:
(a) affinity purifying said unpredicted polypeptide(s) by contacting an antibody that binds to said known polypeptide with said biological sample and removing material not bound by said antibody; and
(b) determining the amino acid sequence or composition of said unpredicted polypeptide(s).

47. A method according to claim 46, wherein said antibody is bound to a solid phase.

48. A method according to claim 46, wherein said antibody is at a discrete location on an antibody array.

49. A method according to claim 46, wherein said amino acid sequence or composition is obtained by mass spectrometry.

50. A method according to claim 49, wherein said amino acid sequence or composition is obtained by SELDI-TOF mass spectrometry.

51. A method according to claim 46, wherein said unpredicted polypeptide(s) are proteolytic fragments of said known polypeptide.

52. A method according to claim 46, further comprising selecting one or more antibodies that distinguish between said known polypeptide and said unpredicted polypeptide(s).

53. A method according to claim 46, further comprising determining if said unpredicted polypeptide(s) are indicative of a disease diagnosis or prognosis.

54. A method according to claim 46, wherein said biological sample is obtained from a disease sample.

55. A method according to claim 54, wherein said method further comprises selecting an antibody that distinguishes one or more of said unpredicted polypeptides from said known polypeptide; and
using said antibody in an immunoassay for one or more of said unpredicted polypeptides.
